(19) United States
(12) Patent Application Publication
(10) Pub. No.: US 2010/0197668 A1
(43) Pub. Date: Aug. 5, 2010

(54) 1,2,3,4-TETRAHYDROPYRROLO[1,2-A]PYRAZINE-6-CARBOXAMIDE AND 2,3,4,5-TETRAHYDROPYRROLO[1,2-a][1,4]-DIAZEPINE-7-CARBOXAMIDE DERIVATIVES, PREPARATION AND THERAPEUTIC USE THEREOF

(75) Inventors: Bernard BAUDOIN, Chaville (FR); Michel EVERS, A Queue En Brie (FR); Arielle GENEVOIS-BORELLA, Thiais (FR); Andreas KARLSSON, Paris (FR); Jean-Luc MALLERON, Marcoussis (FR); Magali MATHIEU, Choisy-Le-Roi (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/693,601

(22) Filed: Jan. 26, 2010

Related U.S. Application Data
(63) Continuation of application No. PCT/FR2008/001096, filed on Jul. 24, 2008.

(30) Foreign Application Priority Data
Jul. 27, 2007 (FR) 0705497
Jul. 27, 2007 (FR) 0705498

(51) Int. Cl. A61K 31/551; C07D 471/04; A61K 31/4985; C07D 487/02; A61P 25/28; A61P 25/16; A61P 25/24; A61P 25/22; A61P 25/06; A61P 3/10; A61P 35/00

(52) U.S. Cl. 514/221; 544/349; 514/249; 540/567

(57) ABSTRACT

The present invention relates to derivatives of 1,2,3,4-tetrahydropyrrolo-[1,2-a]pyrazine-6-carboxamides and of 2,3,4,5-tetrahydropyrrolo[1,2-a][1,4]diazepine-7-carboxamides, to the preparation thereof and to the therapeutic use thereof.

This application is a Continuation of International Application No. PCT/FR2008/001096, filed Jul. 24, 2008, which is incorporated herein by reference in its entirety.

The present invention relates to derivatives of 1,2,3,4-tetrahydropyrrolo-[1,2-a]pyrazine-6-carboxamides and of 2,3,4,5-tetrahydropyrrolo[1,2-a][1,4]diazepine-7-carboxamides, to the preparation thereof and to the therapeutic use thereof.

The presence of multiple senile plaques in brain tissue is one of the main histopathological alterations observed in Alzheimer's disease; these plaques form by deposition of fibrillary aggregates of a 4 kDa peptide (40-42 amino acids), known as amyloid β (Aβ) peptide. The production and gradual accumulation of this peptide could play a crucial role in the triggering and progression of Alzheimer's pathology, according to the amyloid cascade hypothesis (D. Seiko et al. Nature 399A (1999) 23; S. Roggo et al. Top. Med. Chem. 2 (2002) 359; A. Ghosh et al. Curr. Med. Chem. 9 (2002) 1135.

The Aβ peptide originates from the APP (Amyloid Precursor Protein) protein, which may be cleaved by at least three different proteolytic activities: 1) cleavage in the Aβ region by an α-secretase activity (thus preventing the formation of Aβ); 2) cleavage at the N-terminal end of Aβ by a β-secretase activity; 3) cleavage at the C-terminal end of Aβ by a γ-secretase activity. The consecutive cleavage of the APP protein at the β and γ sites leads to the formation of the Aβ peptide (M. Citron Nat. Rev. Neurosci. 5 (2004) 677-685; D. Beher et al. Expert Opin. Invest. Drugs 14 (2005) 1385-1409).

There is therefore a real interest in finding compounds that inhibit the production of the Aβ peptide (T. B. Durham et al. Curr. Opin. Drug Disc. Dev. 9 (2006) 776-791).

It has now been found that compounds, derivatives of 1,2,3,4-tetrahydropyrrolo[1,2-a]pyrazine-6-carboxamides and of 2,3,4,5-tetrahydropyrrolo-[1,2-a][1,4]diazepine-7-carboxamides possess a strong inhibitory activity with respect to the β-secretase activity.

One subject of the present invention is the compounds corresponding to the formula (I)

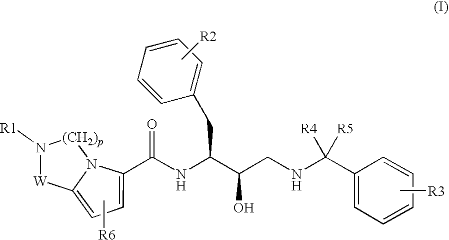

in which:
R1 represents a hydrogen atom, a (C₁-C₁₀)alkyl, (C₃-C₇)cycloalkyl, (CH₂)ₙ—(C₁-C₆)alkenyl, (CH₂)ₙ—(C₁-C₆)alkynyl or (C₁-C₆)alkyl-Z—(C₁-C₆)alkyl group, in which Z represents a heteroatom chosen from O, N and S(O)ₘ, or else R1 represents a COOR, S(O)ₘR, aryl or aralkyl group; (C₁-C₁₀)alkyl, (C₃-C₇)cycloalkyl, (CH₂)ₙ—(C₁-C₆)alkenyl, (CH₂)ₙ—(C₁-C₆)alkynyl, (C₁-C₆)alkyl-Z—(C₁-C₆)alkyl, aryl or aralkyl groups being optionally substituted with one or more groups chosen from a halogen atom, a (C₁-C₆)alkyl, (C₃-C₇)cycloalkyl, halo(C₁-C₆)alkyl, (C₁-C₆)alkoxy, halo(C₁-C₆)alkoxy, NR7R8, nitro, cyano, OR, COOR, C(O)NR7R8 or S(O)ₘNR7R8 group;
R2 represents one or more groups chosen from a hydrogen atom, a halogen atom, a (C₁-C₆)alkyl, (C₃-C₇)cycloalkyl, (C₁-C₆)alkenyl, (C₁-C₆)alkynyl or (C₁-C₆)alkyl-Z—(C₁-C₆)alkyl group, in which Z represents a heteroatom chosen from O, N and S(O)ₘ, or else R² represents a halo(C₁-C₆)alkyl, halo(C₁-C₆)alkoxy, hydroxy, (C₁-C₆)alkoxy, nitro, cyano or amino group, an NR7R8, COOR, C(O)NR7R8, O—C(O)(C₁-C₆)alkyl or S(O)ₘ—NR7R8 group, or an aryl group, the aryl group possibly being optionally substituted with one or more groups chosen from a halogen atom, (Cᵣ C₆)alkyl, (C₃-C₇)cycloalkyl, halo(C₁-C₆)alkyl, (C₁-C₆)alkoxy, halo(C₁-C₆)alkoxy, NR7R8, OR, nitro, cyano, COOR, C(O)NR7R8 or S(O)ₘNR7R8 group;
R3 represents a trifluoromethyl group;
R4 and R5 represent a hydrogen atom, or else R4 and R5 form, with the carbon atom that bears them, a saturated ring containing from 3 to 6 carbon atoms and optionally containing from 0 to 1 heteroatom, chosen from O, N or S;
R6 represents a group chosen from a hydrogen atom, a halogen atom, a (C₁-C₆)alkyl, (C₃-C₇)cycloalkyl, (C₃-C₇)cycloalkyl(C₁-C₆)alkyl, nitro or amino group, an NR7R8 or COOR group, an aryl group, or an NR7(SO₂)R8 or C(O)NR7R8 group;
R, R7 and R8 represent, independently of one another, one or more groups chosen from a hydrogen atom, a (C₁-C₆)alkyl, (C₃-C₇)cycloalkyl or (C₃-C₇)cycloalkyl(C₁-C₆)alkyl group, an aryl or aryl(C₁-C₆)alkylene group, or else R7 and R8 may form, with the atom which bears them, a saturated, partially unsaturated or unsaturated ring containing from 5 to 7 carbon atoms and optionally containing, in addition, a heteroatom chosen from O, N or S(O)ₘ;
W represents a methylene or C(O) group;
m represents an integer which may take the values 0, 1 or 2;
n represents an integer which may take the values 1, 2, 3, 4, 5 or 6;
p represents an integer which may take the values 2 or 3;
the carbon bearing the benzyl group substituted by R2 is of S absolute configuration; and
the carbon bearing the hydroxyl group is of R absolute configuration, in the form of a base or addition salt with an acid.

The compounds of formula (I) may comprise one or more asymmetric carbon atoms. They may therefore exist in the form of enantiomers or of diastereoisomers. These enantiomers or diastereoisomers, and also mixtures thereof, including racemic mixtures, are part of the invention.

The compounds of formula (I) may exist in the form of bases or addition salts with acids. Such addition salts are part of the invention.

These salts may be prepared with pharmaceutically acceptable acids, but the salts of other acids that are useful, for example, for the purification or isolation of the compounds of formula (I) are also part of the invention.

Within the context of the present invention, the following definitions apply:
Cₜ-C_{z}, where t and z may take the values of 1 to 10, is understood to mean a carbon-based chain or ring which may have from t to z carbon atoms, for example C₁-C₃ may characterize a carbon-based chain having from 1 to 3 carbon atoms;
a halogen atom: a fluorine, a chlorine, a bromine or an iodine;
an alkyl group: a linear or branched saturated aliphatic group. By way of examples, mention may be made of the methyl, ethyl, propyl, isopropyl, butyl, isobutyl, tert-butyl, pentyl, etc. groups;
a cycloalkyl group: a cyclic alkyl group. By way of examples, mention may be made of the cyclopropyl, methylcyclopropyl, cyclobutyl, cyclopentyl, cyclohexyl, etc. groups;
an alkylene group: a linear or branched saturated divalent aliphatic group. By way of example, a C₁₋₃-alkylene group represents a linear or branched divalent carbon-based chain having 1 to 3 carbon atoms, such as a methylenyl (—CH₂—), an ethylenyl (—CH₂CH₂—), a 1-methylethylenyl (—CH(CH₃)CH₂—) or a propylenyl (—CH₂CH₂CH₂—);
a haloalkyl group: an alkyl group of which one or more hydrogen atoms have been substituted with a halogen atom. By way of examples, mention may be made of the CF₃, CH₂CF₃, CHF₂ or OCCl₃ groups;
a haloalkoxy group: an alkoxy group of which one or more hydrogen atoms have been substituted with a halogen atom. By way of examples, mention may be made of the OCF₃, OCHF₂ or OCCl₃ groups;
an alkenyl group: a linear or branched monounsaturated or poly-unsaturated aliphatic group comprising, for example, one or two ethylenically unsaturated groups;
an alkenyl group: a linear or branched monounsaturated or poly-unsaturated aliphatic group comprising, for example, one or two acetylenically unsaturated groups;
an alkoxy group: an —O-alkyl radical where the alkyl group is as defined previously;
an aryl group: a cyclic aromatic group comprising between 6 and 14 carbon atoms. By way of example of an aryl group, mention may be made of phenyl or naphthyl;
the sulfur and nitrogen atoms may be present in the oxidized state (N-oxide, sulfoxide, sulfone).
When p represents 2, the compounds correspond to the following formula:

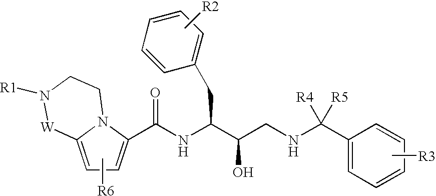

When p represents 3, the compounds correspond to the following formula:

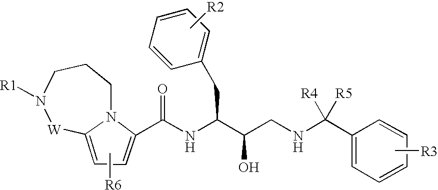

In the various groups as defined below, the groups R1, R2, R3, R4, R5, R6, R, R7, R8, W and p, when they are not defined, have the same definitions as those mentioned above.

Among the compounds of formula (I) that are subjects of the invention, a first group of compounds is constituted by the compounds for which:

W represents a methylene group.

Among the compounds of formula (I) that are subjects of the invention, a second group of compounds is constituted by the compounds for which:

W represents a C(O) group.

Among the compounds of formula (I) that are subjects of the invention, a third group of compounds is constituted by the compounds for which:

p represents 2.

Among the compounds of formula (I) that are subjects of the invention, a fourth group of compounds is constituted by the compounds for which:

p represents 3.

Among the compounds of formula (I) that are subjects of the invention, a fifth group of compounds is constituted by the compounds for which:

R6 represents a group chosen from a hydrogen atom, a COOR group or a C(O)NR7R8 group.

Among this subgroup of compounds of formula (I) that are subjects of the invention, a sixth group of compounds is constituted by the compounds for which:

R6 represents a group chosen from a hydrogen atom, a COOH or COOMe group or a C(O)N(Et)₂ group.

Among the compounds of formula (I) that are subjects of the invention, a seventh group of compounds is constituted by the compounds for which:

W represents a methylene or C(O) group;
p represents 2 or 3;
R1 represents a hydrogen atom, a (C₁-C₁₀)alkyl, COOR or S(O)ₘR group, the (C₁-C₁₀)alkyl group being optionally substituted with one or more (C₁-C₆)alkyl groups;
R2 represents one or more groups chosen from a hydrogen atom or a halogen atom;
R4 and R5 represent a hydrogen atom or form, with the carbon atom which bears them, a cyclopropyl group;
R6 represents a group chosen from a hydrogen atom, a COOR group or a C(O)NR7R8 group; and
R, R7 and R8 represent, independently of one another, a hydrogen atom or one or more (C₁-C₆)alkyl groups.

The combinations of groups 1 to 7 as defined above are also part of the invention.

Among the compounds of formula (I) that are subjects of the invention, mention may also be made of the following compounds:
2-tert-butyl 8-methyl 6-{[(1S,2R)-1-benzyl-2-hydroxy-3-({1-[3-(trifluoromethyl)phenyl]cyclopropyl}amino)propyl]carbamoyl}-3,4-dihydropyrrolo[1,2-a]pyrazine-2,8(1H)-dicarboxylate;
6-{[(1S,2R)-1-benzyl-2-hydroxy-3-({1-[3-(trifluoromethyl)phenyl]cyclopropyl}amino)propyl]carbamoyl}-2-(tert-butoxycarbonyl)-1,2,3,4-tetrahydropyrrolo[1,2-a]pyrazine-8-carboxylic acid;
6-{[(1S,2R)-1-benzyl-2-hydroxy-3-({1-[3-(trifluoromethyl)phenyl]cyclopropyl}amino)propyl]carbamoyl}-1,2,3,4-tetrahydropyrrolo[1,2-a]pyrazine-8-carboxylic acid hydrochloride (2:1);
tert-butyl 6-{[(1S,2R)-1-benzyl-2-hydroxy-3-({1-[3-(trifluoromethyl)phenyl]cyclopropyl}amino)propyl]carbamoyl}-8-(diethylcarbamoyl)-3,4-dihydropyrrolo[1,2-a]pyrazine-2(1H)carboxylate;
6-{(1S,2R)-1-benzyl-2-hydroxy-3-[1-(3-trifluoromethylphenyl)cyclopropylamino]propyl}-N-8,N-8-diethyl-1,2,3,4-tetrahydropyrrolo[1,2-a]pyrazine-6,8-dicarboxamide and its hydrochloride (2:1);
6-{(1S,2R)-1-benzyl-2-hydroxy-3-[1-(3-trifluoromethylphenyl)cyclopropylamino]propyl}-N-8,N-8-diethyl-2-(methylsulfonyl)-1,2,3,4-tetrahydropyrrolo[1,2-a]pyrazine-6,8-dicarboxamide;
N-[(1S,2R)-1-(3,5-difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide and its hydrochloride (1:1);
N-[(1S,2R)-1-benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide and its hydrochloride (1:1);
N-[(1S,2R)-1-benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide and its hydrochloride (1:1);
N-[(1S,2R)-1-benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide and its hydrochloride (1:1); and
N-[(1S,2R)-1-benzyl-2-hydroxy-3-{[3-(trifluoromethyl)benzyl]amino}propyl]-1-oxo-2-(1-propylbutyl)-2,3,4,5-tetrahydro-1H-pyrrolo[1,2-a][1,4]diazepine-7-carboxamide and its hydrochloride (1:1).

Another subject of the invention is a method for preparing compounds of the invention of formula (I).

In what follows, the expression “protective group Pg” is understood to mean a group which makes it possible, on the one hand, to protect a reactive functional group such as a hydroxyl or an amine during a synthesis and, on the other hand, to regenerate the reactive functional group intact at the end of the synthesis. Examples of protective groups and also of methods of protection and of deprotection are given in “Protective Groups in Organic Synthesis”, Green et al., 2^{nd} Edition (John Wiley & Sons, Inc., New York), 1991.

The expression “leaving group” is understood to mean, in what follows, a group which may be easily cleaved from a molecule by rupture of a heterolytic bond, with departure of an electron pair. This group may thus be easily replaced by another group during a substitution reaction for example. Such leaving groups are, for example, halogens or an activated hydroxyl group such as a methanesulfonate, benzenesulfonate, p-toluenesulfonate, triflate, acetate, etc. Examples of leaving groups and also references for the preparation thereof are given in “Advances in Organic Chemistry”, J. March, 3^{rd} Edition, Wiley Interscience, 1985, p. 310-316.

In the schemes which follow, the starting compounds and the reactants, when their method of preparation is not described, are commercially available or are described in the literature, or else may be prepared according to methods which are described therein or which are known to a person skilled in the art.

The abbreviations and symbols used for the description of the methods of synthesis and for the description of the compounds are the following:
BOC for tert-butoxycarboxylate,
DCC for dicyclohexylcarbodiimide,
DMF for dimethylformamide,
EDCI for (1-ethyl-3,3-dimethylaminopropyl)carbodiimide,
NMP for N-methyl-2-pyrrolidone,
PyBOP for benzotriazol-1-yloxytripyrrolidinophosphonium hexafluorophosphate,
THF for tetrahydrofuran.

In accordance with the invention, it is possible to prepare the compounds of general formula (I) according to the method illustrated by scheme 1 below.

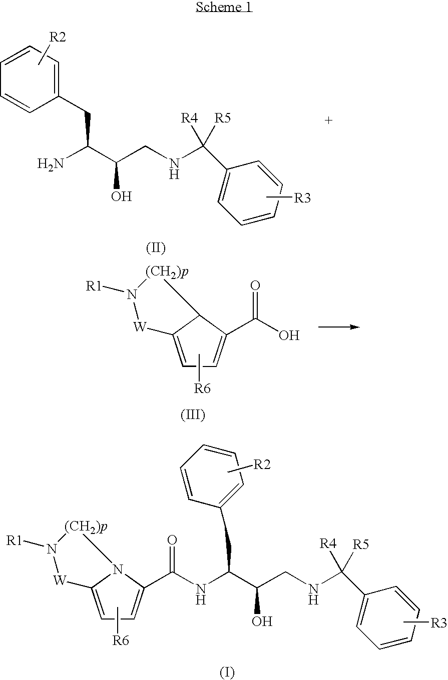

The compound of general formula (I) may be prepared by condensation of the amine functional group of the compound of general formula (II), in which R2, R3, R4 and R5 are as defined in the general formula (I), with the carboxylic acid functional group of the compound of general formula (III), in which p, R1 and R6 are as defined in the general formula (I). This reaction is carried out in an anhydrous medium that is preferably inert (nitrogen or argon for example) and by using conventional agents for coupling an acid functional group with an amine functional group such as DCC, PyBOP or EDCI, in solvents such as dichloromethane, THF, ether or chloroform at a temperature between 20° C. and the reflux temperature of the solvent.

The compound of general formula (II) in which R2, R3, R4 and R5 are as defined in the general formula (I), may be prepared from the compound of general formula (IV), in which R2, R3, R4 and R5 are as defined in the general formula (I), by deprotection of the primary amine by action of an acid (for example hydrochloric acid) in solution in a solvent or a solvent mixture that is etherated (for example diethyl ether) and/or chlorinated (for example dichloromethane), according to the method illustrated by scheme 2 below.

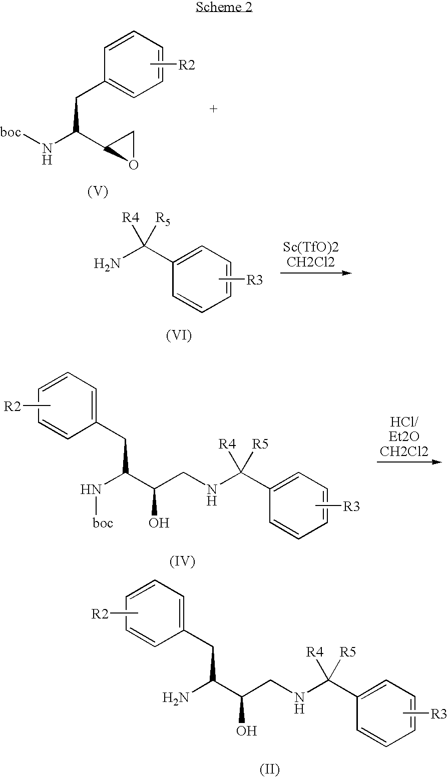

The compound of general formula (IV), in which R2, R3, R4 and R5 are as described previously, may be prepared by reacting a benzylamine derivative of general formula (VI), in which R3, R4 and R5 are as defined in the general formula (I) with an oxirane of general formula (V), in which R2 is as defined in the general formula (I) by operating in an anhydrous medium that is preferably inert (nitrogen or argon for example), in a chlorinated solvent (for example dichloromethane) and in the presence of a Lewis acid such as, for example, scandium triflate.

The compounds of general formula (I) in which W represents C(O) and p represents 2, may be obtained by condensation of the amine functional group of the compound of general formula (II) with the carboxylic acid functional group of the compound of general formula (IIIa), in which R1 and R6 are as described previously.

The compounds of general formula (IIIa) may be prepared according to the method of scheme 3 below, from the compound of general formula (VII), in which R1 and R6 are as defined previously, by reacting the compound of general formula (VII) with carbon monoxide (under a pressure of 1 to 20 atmospheres), in the presence of (potassium or sodium) acetate ions, and an alkali metal iodide (sodium or potassium iodide for example), in the presence of a palladium catalyst (palladium acetate for example), of a phosphine (triphenylphosphine for example) in solution in an organic solvent (dimethylformamide for example) and in the presence of water. The reaction takes place at a temperature between 20° C. and that of the reflux of the solvent.

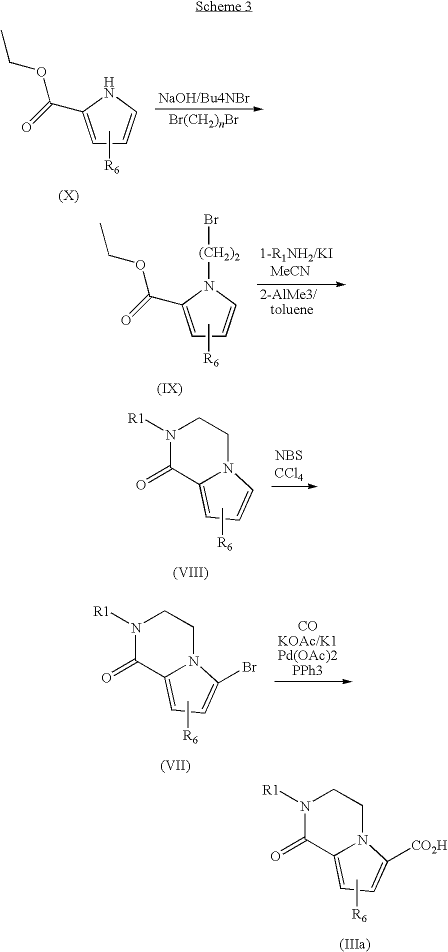

The halogenated derivative of general formula (VII), in which R1 and R6 are as described previously, may be prepared from the bicyclic derivative of general formula (VIII), by reaction with a halogenating agent such as N-bromosuccinimide in a chlorinated solvent (carbon tetrachloride for example).

The bicyclic derivative of general formula (VIII) may be prepared by aminolysis of the compound of general formula (IX) with an amine of general formula R₁NH₂. This reaction is carried out in the presence or absence of iodide ions (potassium or sodium iodide for example) and in solution in an organic solvent (acetonitrile, dimethylformamide, ethanol, NMP, ether, THF, dioxane or toluene for example), at a temperature between 20° C. and the reflux temperature of the solvent. The amine derivative obtained intermediately is then cyclized in the presence of a cyclization agent (trimethylaluminum for example) in solution in an organic solvent (toluene for example) at a temperature between 20° C. and the reflux temperature of the solvent.

The compound of general formula (IX) may be prepared from the ethyl pyrrole-2-carboxylate derivative of general formula (X) by a monoalkylation reaction, by action of 1,2-dibromoethane. This alkylation is carried out with a base such as cesium carbonate, potassium carbonate, potassium phosphate, potassium t-butylate, sodium hydroxide, or sodium hydride and in solvents such as ethanol, DMF, NMP, ether, THF, dioxane, acetonitrile or toluene or by phase transfer in the presence of an agent such as tetrabutylammonium chloride in dichloromethane or dichloroethane in the presence of water, or else in benzene.

The compounds of formula (I) in which W represents a methylene group and p represents 2, may be obtained by condensation of the amine functional group of the compound of general formula (II) with the carboxylic acid functional group of the compound of general formula (IIIb), according to the method of scheme 4 below.

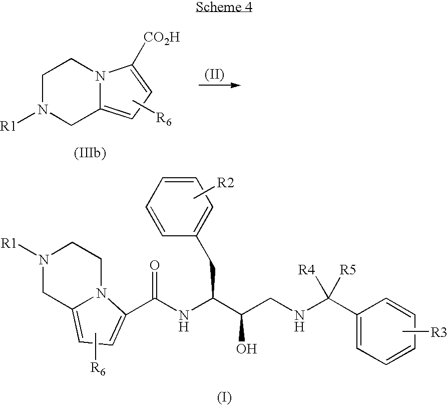

Among the compounds of general formula (IIIb), the compound of formula (IIIb′), in which R6 represents COOR, R being as defined previously, is particularly advantageous for the synthesis of compounds of formula (I). This compound of formula (IIIb′) (2-(tert-butoxycarbonyl)-8-(methoxycarbonyl)-1,2,3,4-tetrahydropyrrolo[1,2-a]pyrazine-6-carboxylic acid) may be synthesized according to the method of scheme 5 below, starting from 2-tert-butyl 8-methyl 6-bromo-3,4-dihydropyrrolo[1,2-a]pyrazine-2,8(1H)-dicarboxylate, under the same conditions as those used for the preparation of the compound of general formula (IIIa).

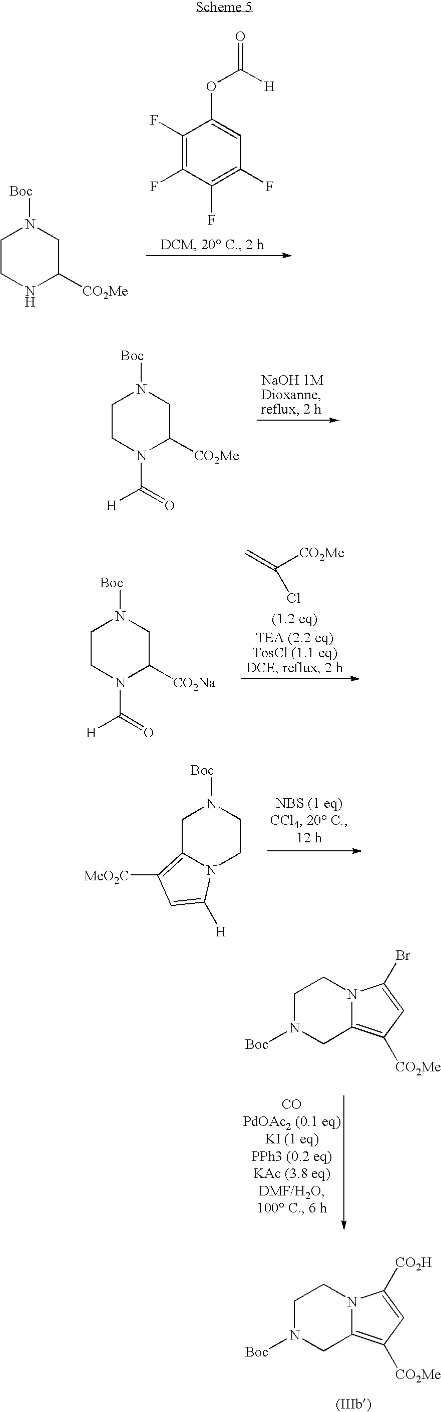

The 2-tert-butyl 8-methyl 6-bromo-3,4-dihydropyrrolo[1,2-a]pyrazine-2,8(1H)dicarboxylate may be prepared by a halogenation reaction of 2-tert-butyl 8-methyl 3,4-dihydropyrrolo[1,2-a]pyrazine-2,8(1H)-dicarboxylate with a halogenating agent such as N-bromosuccinimide, in a chlorinated solvent (carbon tetrachloride for example) at a temperature between 0° C. and the reflux temperature of the solvent.

The 2-tert-butyl 8-methyl 3,4-dihydropyrrolo[1,2-a]pyrazine-2,8(1H)-dicarboxylate may be prepared from sodium 4-(tert-butoxycarbonyl)-1-formylpiperazine-2-carboxylate reacted with a mixture of tosyl chloride and of methyl α-chloroacrylate in the presence of an organic base such as triethylamine or pyridine, in a chlorinated solvent such as dichloromethane or dichloroethane at a temperature between 20° C. and the reflux temperature of the solvent.

The sodium 4-(tert-butoxycarbonyl)-1-formylpiperazine-2-carboxylate may be prepared from commercial 2-methoxycarbonyl-4-N-tert-butyl piperazine, by a formylation reaction with pentafluorophenyl formate (which is prepared extemporaneously according to L. Kisfaludy et al., Synthesis (1987), 5, 510) by operating in an anhydrous medium that is preferably inert (nitrogen or argon for example) in a chlorinated organic solvent (dichloromethane for example) at a temperature close to 20° C., followed by a salification reaction by action of a base such as sodium hydroxide, potassium carbonate or potassium tert-butylate in solvents such as THF or dioxane, at a temperature close to 20° C.

The compounds of general formula (I) in which W represents a CO group and p represents 3, may be obtained by condensation of the amine functional group of the compound of general formula (II) with the carboxylic acid functional group of the compound of general formula (IIIc), the latter being obtained according to the method of scheme 6 below.

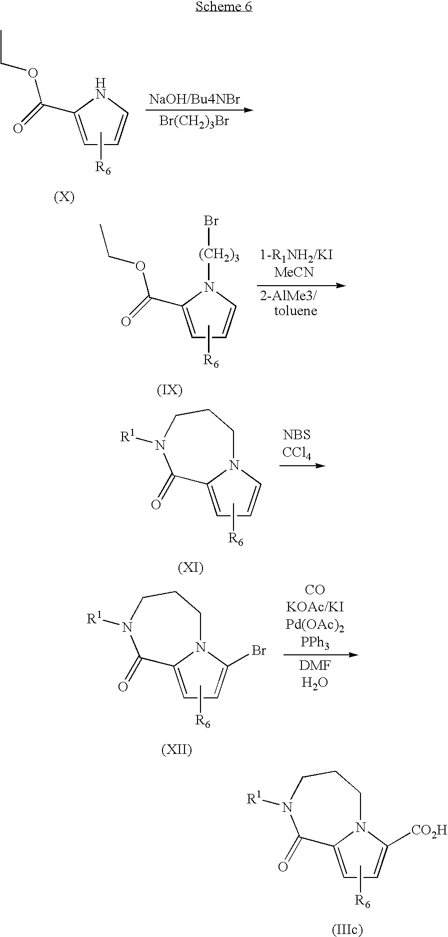

The compounds of general formula (IIIc) may be prepared by reacting the compound of general formula (XII) with carbon monoxide, in the presence of (potassium or sodium) acetate ions, and of an alkali metal iodide (sodium or potassium iodide for example), in the presence of a palladium catalyst (palladium acetate for example), of a phosphine (triphenylphosphine for example) in solution in an organic solvent (dimethylformamide for example) and in the presence of water. The reaction takes place at a temperature between 20° C. and that of the reflux of the solvent. The halogenated derivative of general formula (XII), in which R1 and R6 are as described previously, may be prepared from the derivative of general formula (XI), by reaction with a halogenating agent such as N-bromosuccinimide in a chlorinated solvent (carbon tetrachloride for example).

The bicyclic derivative of general formula (XI) may be prepared by aminolysis of the compound of general formula (IX) with an amine of general formula R1-NH₂. This reaction is carried out in the presence or absence of iodide ions (potassium or sodium iodide for example) and in solution in an organic solvent (acetonitrile, dimethylformamide, ethanol, NMP, ether, THF, dioxane or toluene for example), at a temperature between 20° C. and the reflux temperature of the solvent. The amine derivative obtained intermediately is then cyclized in the presence of a cyclization agent (trimethylaluminum for example) in solution in an organic solvent (toluene for example) at a temperature between 20° C. and the reflux temperature of the solvent.

The compound of general formula (IX) may be prepared from the ethyl pyrrole-2-carboxylate derivative of general formula (X) by a monoalkylation reaction, by action of 1,3-dibromopropane. This alkylation is carried out with a base such as cesium carbonate, potassium carbonate, potassium phosphate, potassium t-butylate, sodium hydroxide, or sodium hydride and in solvents such as ethanol, DMF, NMP, ether, THF, dioxane, acetonitrile or toluene or by phase transfer in the presence of an agent such as tetrabutylammonium chloride in dichloromethane or dichloroethane in the presence of water, or else in benzene.

The products of formula (I) may be subjected, if desired and if necessary, in order to obtain products of formula (I) or to be converted into other products of formula (I), to one or more of the following conversion reactions, in any order:
a) an esterification or amidification reaction of an acid functional group;
b) a hydrolysis reaction of an ester functional group to an acid functional group;
c) a reaction for conversion of a hydroxyl functional group to an alkoxy functional group;
d) an oxidation reaction of an alcohol functional group to an aldehyde, ketone or acid functional group;
e) a reduction reaction of an acid, aldehyde or ketone functional group to an alcohol functional group;
f) a reductive amination reaction of an aldehyde or ketone functional group;
g) an oxidation reaction of an alkenyl group to an aldehyde or ketone functional group;
h) an oxidation reaction of a thioether to a sulfone or sulfoxide;
i) an alkylation reaction of a sulfonamide;
j) a dehydration reaction of a hydroxyalkyl group to an alkenyl group;
k) a dehydrohalogenation reaction of a halogenated derivative;
l) a total or partial hydrogenation reaction of an alkenyl or alkynyl group to an alkenyl or alkyl group;
m) a catalytic coupling reaction of a halogenated derivative and of an organometallic derivative such as a stannic or boronic derivative in order to introduce an alkyl, alkenyl, alkynyl or aryl substituent;
n) a reaction for protecting reactive functional groups;
o) a reaction for eliminating protective groups that the protected reactive functional groups may bear;
p) a salification reaction by a mineral or organic acid or by a base in order to obtain the corresponding salt;
q) a reaction for resolution of racemic forms into enantiomers, said products of formula (I) thus obtained being, where appropriate, in all the possible racemic, enantiomeric and diastereoisomeric isomer forms;
r) a reduction reaction of nitro derivatives to nitroso or amino derivatives;
s) a monoalkylation or dialkylation reaction of an amine functional group;
t) a sulfonylation reaction of a primary or secondary amine; and
u) an acylation reaction of an amine functional group.

Another subject of the invention is, according to another of its aspects, the compounds of formulae (IIIa), (IIIb), (IIIb′) and (IIIc). These compounds are useful as synthesis intermediates of the compounds of formula (I).

The compounds of formula (I) may be purified by methods known to a person skilled in the art, for example by crystallization, chromatography or extraction.

The following examples describe the preparation of certain compounds in accordance with the invention. These examples are not limiting and serve only to illustrate the present invention. The numbers of the compounds exemplified refer to those given in the table below, which illustrates the chemical structures and the physical properties of a few compounds according to the invention.

The proton nucleomagnetic resonance (¹H NMR) spectra were performed at 250 MHz, 300 MHz, 400 MHz or 500 MHz on Bruker machines (chemical shifts (δ in ppm)—in the solvent dimethylsulfoxide—d₆ (DMSO-d₆) referenced to 2.50 ppm at the temperature of 303K). The abbreviations used for characterizing the signals are the following: s=singlet, m=multiplet, d=doublet, t=triplet, q=quadruplet.

The nomenclature of the exemplified compounds below was established using ACDLabs® version 10.0 software.

### EXAMPLE 1

### 2-tert-Butyl 8-methyl 6-{[(1S,2R)-1-benzyl-2-hydroxy-3-({1-[3-(trifluoromethyl)phenyl]cyclopropyl}amino)propyl]carbamoyl}-3,4-dihydropyrrolo[1,2-a]pyrazine-2,8(1H)-dicarboxylate

The preparation of pentafluorophenyl formate is described in the literature (Lajos Kisfaludy et al., Synthesis 1987, 5, 510).

### 1.1: 1-tert-Butyl 3-methyl 4-formylpiperazine-1,3-dicarboxylate

10 g of 2-methoxycarbonyl-4-N-tert-butyl piperazine are dissolved in 25 cm³ of dichloromethane under an inert atmosphere at a temperature close to 20° C. The pentafluorophenyl formate solution obtained in the preceding step is added dropwise at a temperature close to 20° C. Stirring is continued for 1 h 30 min after the end of the addition. The reaction mixture is concentrated using a rotary evaporator under reduced pressure (5 kPa). The yellow-orange oil obtained is purified by flash chromatography through a silica cartridge (column: 700 g; particle size: 40-60 μm; flow rate: 80 cm³/min; eluent: 30% cyclohexane/70% ethyl acetate). After concentrating the fractions under reduced pressure (5 kPa), 10.5 g of 1-tert-butyl 3-methyl 4-formylpiperazine-1,3-dicarboxylate are obtained in the form of a pale yellow oil.

NMR: for this batch, a 50%-50% resolution of rotamers is observed with:

1.38 (s, 4.5H); 1.39 (s, 4.5H); from 2.62 to 2.93 (m, 1.5H); 3.08 (m, 1H); 3.26 (partially masked m, 0.5H); 3.64 (partially masked m, 0.5H); 3.68 (s, 1.5H); 3.69 (s, 1.5H); 3.90 (m, 1H); 4.02 (m, 0.5H); 4.36 (broad d, J=13.5 Hz, 0.5H); 4.42 (broad d, J=13.5 Hz, 0.5H); 4.71 (broad d, J=4.5 Hz, 0.5H); 4.89 (broad d, J=4.5 Hz, 0.5H); 8.09 (s, 0.5H); 8.16 (s, 0.5H).

### 1.2: Sodium 4-(tert-butoxycarbonyl)-1-formylpiperazine-2-carboxylate

10.5 g of 1-tert-butyl 3-methyl 4-formylpiperazine-1,3-dicarboxylate are dissolved in 200 cm³ of dioxane at a temperature close to 20° C. 42 cm³ of a 1M sodium hydroxide solution are added, then the reaction mixture is heated at the reflux of the solvent for 1 h 30 min. The reaction mixture is concentrated using a rotary evaporator under reduced pressure (5 kPa). The solid beige residue obtained is triturated in ethyl acetate, then filtered, rinsed and dried in the air. 10.5 g of sodium 4-(tert-butoxycarbonyl)-1-formylpiperazine-2-carboxylate are obtained in the form of an amorphous white solid.

LC-MS-DAD-ELSD: 259⁽⁺⁾=(M+H)⁽⁺⁾

NMR: 8.08 (d, 1H); 4.74 (d, 0.5H); 4.55 (m, 1H); 4.39 (d, 0.5H); 4.00 (dd, 1H); 3.62 (d, 0.5H); 3.43 (t, 0.5H); 3.00 (m, 3H); 1.40 (s, 9H).

### 1.3: 2-tert-Butyl 8-methyl 3,4-dihydropyrrolo[1,2-a]pyrazine-2,8(1H)-dicarboxylate

10.5 g of sodium 4-(tert-butoxycarbonyl)-1-formylpiperazine-2-carboxylate are dissolved in 300 cm³ of dichloroethane at a temperature close to 20° C. 8.5 g of tosyl chloride, 5.3 cm³ of methyl α-chloroacrylate and 12.6 cm³ of triethylamine are added. The reaction mixture is heated at the reflux of the solvent for 1 h. The reaction mixture is cooled to a temperature close to 20° C. It is then washed successively with two lots of 100 cm³ of water and with two lots of 100 cm³ of a saturated aqueous solution of sodium chloride. The aqueous phases are reextracted using dichloromethane. The organic phases are combined, dried over a phase separator filter, then concentrated using a rotary evaporator under reduced pressure (5 kPa). The brown-orange oil obtained is purified by flash chromatography through a silica cartridge (column: 700 g; particle size: 40-60 μm; flow rate 80 cm³/min; eluant: 80% cyclohexane/20% ethyl acetate). After concentrating the fractions under reduced pressure (5 kPa), 9.5 g of 2-tert-butyl 8-methyl 3,4-dihydropyrrolo[1,2-a]pyrazine-2,8(1H)-dicarboxylate are obtained in the form of a pale yellow solid.

NMR (ppm): 6.77 (d, 1H); 6.42 (d, 1H); 4.73 (s, 2H); 3.98 (t, 2H); 3.72 (t, 2H); 3.70 (s, 3H); 1.43 (s, 9H)

LC-MS-DAD-ELSD: 281⁽⁺⁾=(M+H)⁽⁺⁾

MP: 100° C.

### 1.4: 2-tert-Butyl 8-methyl 6-bromo-3,4-dihydropyrrolo[1,2-a]pyrazine-2,8(1H)-dicarboxylate

9.5 g of 2-tert-butyl 8-methyl 3,4-dihydropyrrolo[1,2-a]pyrazine-2,8(1H)-dicarboxylate are dissolved in 1 dm³ of carbon tetrachloride at a temperature close to 20° C. The reaction mixture is cooled to 4° C. using an ice/water bath and 6 g of N-bromosuccinimide are added in two lots at min intervals. Stirring is continued for 18 h, during which the reaction mixture gradually rises to a temperature close to 20° C. 6 g of sodium hydrogen carbonate and 6 g of sodium sulfate dissolved in 150 cm³ of water are added to the reaction mixture (pH 9). The organic phase is successively washed with 150 cm³ of water then with 150 cm³ of a saturated aqueous solution of sodium chloride. The aqueous phases are reextracted using carbon tetrachloride. The organic phases are combined, dried over a phase separator filter then concentrated using a rotary evaporator under reduced pressure (5 kPa). The yellow oil obtained is triturated in cyclohexane. The brominated derivative which has precipitated is filtered and rinsed using cyclohexane. 8.6 g of 2-tert-butyl 8-methyl 6-bromo-3,4-dihydropyrrolo[1,2-a]pyrazine-2,8(1H)-dicarboxylate are obtained in the form of a pale yellow solid.

NMR: 6.57 (s, 1H); 4.72 (s, 2H); 3.88 (t, 2H); 3.76 (t, 2H); 3.71 (s, 3H); 1.43 (s, 9H)

LC-MS-DAD-ELSD: 359⁽⁺⁾⁷⁹Br=(M+H)⁽⁺⁾, 381⁽⁺⁾⁷⁹Br=(M+Na)⁽⁺⁾

MP: 109° C.

### 1.5: 2-(tert-Butoxycarbonyl)-8-(methoxycarbonyl)-1,2,3,4-tetrahydropyrrolo[1,2-a]pyrazine-6-carboxylic acid

8.6 g of 2-tert-butyl 8-methyl 6-bromo-3,4-dihydropyrrolo[1,2-a]pyrazine-2,8(1H)-dicarboxylate are dissolved in 320 cm³ of dimethylformamide at a temperature close to 20° C. 9 g of potassium acetate and 4.2 g of potassium iodide are added, then the reaction mixture is purged using carbon monoxide. Under a carbon monoxide atmosphere, 20 cm³ of water are added and also 538 mg of palladium acetate and 1.3 g of triphenylphosphine. The reaction mixture is subjected to carbon monoxide bubbling, then is heated at 100° C. for 6 h. It is then concentrated using a rotary evaporator under reduced pressure (5 kPa). The solid residue obtained is taken up in 300 cm³ of ethyl acetate and 220 cm³ of sodium hydroxide are added, respectively 200 cm³ of 1M and 20 cm³ of 5M (pH 12). After filtration of the palladium and decantation of the filtrate, the acid is precipitated by addition of 70 cm³ of 5M hydrochloric acid to the aqueous phase. The solid is filtered and dried in the air. 6.9 g of 2-(tert-butoxy-carbonyl)-8-(methoxycarbonyl)-1,2,3,4-tetrahydropyrrolo[1,2-a]pyrazine-6-carboxylic acid are obtained in the form of a white solid.

NMR: 7.09 (s, 1H); 4.79 (s, 2H); 4.32 (t, 2H); 3.73 (m, 5H); 1.44 (s, 9H)

LC-MS-DAD-ELSD: 325⁽⁺⁾=(M+H)⁽⁺⁾; 225⁽⁺⁾=(M+H)⁽⁺⁾-t-BOC+H

MP: 177° C.

The preparation of 1-(3-trifluoromethyl)phenylcyclopropylamine is described in the literature (Armin de Meijere et al., Organic Letters 2003, 5(5), 753-755).

### 1.6: tert-Butyl[(1S,2R)-1-benzyl-2-hydroxy-3-({1-[3-(trifluoromethyl)phenyl]-cyclopropyl}amino)propyl]carbamate

4.6 g of 1-(3-trifluoromethyl)phenylcyclopropylamine are dissolved in 12 cm³ of dichloromethane at a temperature close to 20° C. 7.9 g of tert-butyl [S—(R,R)]-(−)-1-oxiranyl-2-phenylethyl)carbamate and 2.3 g of scandium triflate are added. The reaction mixture is kept stirring at 20° C. for 12 h. It is then diluted in 100 cm³ of dichloromethane and washed successively with two lots of 15 cm³ of water, 20 cm³ of a saturated aqueous solution of sodium hydrogen carbonate and with 50 cm³ of a saturated aqueous solution of sodium chloride. The aqueous phases are extracted using dichloromethane, and the organic phases are combined, dried over a phase separator filter and concentrated using a rotary evaporator under reduced pressure (5 kPa). The white solid obtained is purified by flash chromatography through a silica cartridge (column: 600 g; particle size: 40-60 μm; flow rate: 80 cm³/min; eluant: 80% diisopropyl ether/20% ethyl acetate). 6.8 g of tert-butyl[(1S,2R)-1-benzyl-2-hydroxy-3-({1-[3-(trifluoromethyl)phenyl]cyclopropyl}amino)propyl]carbamate are obtained in the form of a white solid.

NMR: 7.64 (s, 1H); 7.53 (m, 3H); 7.23 (m, 2H); 7.15 (m, 3H); 6.56 (d, 1H); 6.12 (d, 1H); 4.70 (d, 1H); 3.51 (m, 1H); 3.38 (m, 1H); 3.18 (m, 1H); 2.98 (dd, 1H); 2.50 (m, 2H); 1.21 (s, 9H); 0.98 (m, 4H)

LC-MS-DAD-ELSD: 465⁽⁺⁾=(M+H)⁽⁺⁾

MP: 124° C.

### 1.7: (2R,3S)-3-Amino-4-phenyl-1-({1-[3-(trifluoromethyl)phenyl]cyclopropyl}amino)butan-2-ol hydrochloride (2:1)

6.8 g of tert-butyl[(1S,2R)-1-benzyl-2-hydroxy-3-({1-[3-(trifluoromethyl)phenyl]cyclopropyl}amino)propyl]carbamate are dissolved in 250 cm³ of dichloromethane at a temperature close to 20° C. 36.6 cm³ of a 4M solution of hydrochloric acid in dioxane are added. The reaction mixture is kept stirring for 1 h 30 min at a temperature close to 20° C. The reaction mixture is concentrated using a rotary evaporator under reduced pressure (5 kPa). The beige solid obtained is triturated in diisopropyl ether then filtered. 5.4 g of (2R,3S)-3-amino-4-phenyl-1-({1-[3-(trifluoromethyl)phenyl]-cyclopropyl}amino)butan-2-ol hydrochloride (2:1) are obtained.

NMR: 1.16 (m, 1H); 1.27 (m, 1H); 1.60 (m, 2H); 2.62 (m, 1H); 2.85 (d, J=7.0 Hz, 2H); 2.95 (m, 1H); 3.52 (m, 1H); 4.15 (m, 1H); 6.17 (m, 1H); 7.24 (m, 5H); 7.65 (t, J=7.5 Hz, 1H); 7.77 (d, J=7.5 Hz, 1H); 7.81 (d, J=7.5 Hz, 1H); 7.94 (s, 1H); 8.23 (broad m, 3H); 9.72 (broad unresolved m, 1H); 10.35 (broad unresolved m, 1H).

LC-MS-DAD-ELSD: 409⁽⁺⁾=(M+Formic Ac-H)⁽⁻⁾, 365⁽⁺⁾=(M⁺)

### 1.8: 2-tert-Butyl 8-methyl 6-{[(1S,2R)-1-benzyl-2-hydroxy-3-({1-[3-(trifluoromethyl)phenyl]cyclopropyl}amino)propyl]carbamoyl}-3,4-dihydropyrrolo[1,2-a]pyrazine-2,8(1H)-dicarboxylate

3.9 g of 2-(tert-butoxycarbonyl)-8-(methoxycarbonyl)-1,2,3,4-tetrahydropyrrolo[1,2-a]pyrazine-6-carboxylic acid and 5.4 g of (2R,3S)-3-amino-4-phenyl-1-({1-[3-(trifluoromethyl)phenyl]cyclopropyl}amino)butan-2-ol hydrochloride (2:1) are dissolved in 270 cm³ of dichloromethane under an inert atmosphere at a temperature close to 20° C. 184 mg of hydroxybenzotriazole and 7.1 cm³ of N,N-diisopropylethylamine are added, followed by 3.1 g of 1-(3-dimethylaminopropyl)-3-ethylcarbodiimide hydrochloride and 166 mg of 4-dimethylaminopyridine. The reaction mixture is kept stirring for 3 h at a temperature close to 20° C. The reaction mixture is washed successively with 50 cm³ of water, two lots of 50 cm³ of a saturated aqueous solution of potassium dihydrogen phosphate, again with 50 cm³ of water and with 100 cm³ of a saturated aqueous solution of sodium chloride. The aqueous phases are extracted using dichloromethane. The organic phases are combined, dried over a phase separator filter and concentrated using a rotary evaporator under reduced pressure (5 kPa). The beige foam obtained is purified by reprecipitation in diisopropyl ether at a temperature close to 20° C. The precipitate is filtered, rinsed with diisopropyl ether and dried in the air. 6.8 g of 2-tert-butyl 8-methyl 6-{[(1S,2R)-1-benzyl-2-hydroxy-3-({1-[3-(trifluoromethyl)phenyl]cyclopropyl}amino)propyl]-carbamoyl}-3,4-dihydropyrrolo[1,2-a]pyrazine-2,8(1H)-dicarboxylate are obtained in the form of a white solid.

NMR: 7.93 (d, 1H); 7.72 (s, 1H); 7.53 (m, 1H); 7.46 (m, 2H); 7.18 (m, 4H); 7.13 (s, 1H); 7.11 (m, 1H); 4.80 (d, 1H); 4.74 (q, 2H); 4.09 (m, 2H); 4.01 (m, 1H); 3.73 (s, 3H); 3.62 (m, 2H); 3.50 (m, 1H); 3.03 (dd, 1H); 2.69 (dd, 1H); 2.50 (m, 2H); 1.42 (s, 9H); 0.80 (m, 4H)

LC-MS-DAD-ELSD: 669⁽⁻⁾=(M−H)⁽⁻⁾; 671⁽⁺⁾=(M+H)⁽⁺⁾

MP: 136° C.

### EXAMPLE 2

### 6-{[(1S,2R)-1-Benzyl-2-hydroxy-3-({1-[3-(trifluoromethyl)phenyl]cyclopropyl}amino)propyl]carbamoyl}-2-(tert-butoxycarbonyl)-1,2,3,4-tetrahydropyrrolo[1,2-a]pyrazine-8-carboxylic acid

2 g of 2-tert-butyl 8-methyl 6-{[(1S,2R)-1-benzyl-2-hydroxy-3-({1-[3-(trifluoromethyl)phenyl]cyclopropyl}amino)propyl]carbamoyl}-3,4-dihydropyrrolo[1,2-a]pyrazine-2,8(1H)-dicarboxylate (prepared in example 1.8) are dissolved in 20 cm³ of methanol at a temperature close to 20° C. 6.8 cm³ of 1M sodium hydroxide are added and the reaction mixture is heated for 18 h at the reflux of the solvent. The methanol of the reaction mixture is evaporated under reduced pressure (5 kPa), then the aqueous residue obtained is acidified by the addition of 6.8 cm³ of 1M hydrochloric acid. The gummy residue obtained is concentrated to dryness using a rotary evaporator, then taken up in ethyl acetate. The sodium chloride salts are filtered, then the filtrate is concentrated using a rotary evaporator under reduced pressure (5 kPa). The beige foam obtained is triturated in diisopropyl ether. The solid obtained is filtered, rinsed with diisopropyl ether and dried in the air. 1.8 g of 6-{[(1S,2R)-1-benzyl-2-hydroxy-3-({1-[3-(trifluoromethyl)phenyl]cyclopropyl}amino)propyl]carbamoyl}-2-(tert-butoxycarbonyl)-1,2,3,4-tetrahydropyrrolo[1,2-a]pyrazine-8-carboxylic acid are obtained in the form of a white powder.

NMR: from 0.87 to 1.06 (m, 4H); 1.42 (s, 9H); 2.46 (dd, J=7.5 and 12.0 Hz, 1H); 2.55 (dd, J=3.5 and 12.0 Hz, 1H); 2.69 (dd, J=10.5 and 14.0 Hz, 1H); 3.04 (dd, J=3.5 and 14.0 Hz, 1H); 3.49 (m, 1H); 3.62 (m, 2H); from 3.96 to 4.15 (m, 3H); 4.73 (m, 2H); 4.80 (m, 1H); 7.09 (s, 1H); 7.11 (m, 1H); 7.19 (m, 4H); 7.42 (m, 2H); 7.54 (m, 1H); 7.61 (broad s, 1H); 7.92 (d, J=9.0 Hz, 1H).

LC-MS-DAD-ELSD: 655⁽⁻⁾=(M−H)⁽⁻⁾; 657⁽⁺⁾=(M+H)⁽⁺⁾

MP: 134.5° C.

### EXAMPLE 3

### 6-{[(1S,2R)-1-Benzyl-2-hydroxy-3-({1-[3-(trifluoromethyl)phenyl]cyclopropyl}amino)propyl]carbamoyl}-1,2,3,4-tetrahydropyrrolo[1,2-a]pyrazine-8-carboxylic acid hydrochloride (2:1)

200 mg of 6-{[(1S,2R)-1-benzyl-2-hydroxy-3-({1-[3-(trifluoromethyl)phenyl]-cyclopropyl}amino)propyl]carbamoyl}-2-(tert-butoxycarbonyl)-1,2,3,4-tetrahydropyrrolo[1,2-a]pyrazine-8-carboxylic acid (prepared as in example 2) are dissolved in 5 cm³ of dichloromethane at a temperature close to 20° C. 0.76 cm³ of a 4M hydrochloric acid solution in dioxane are added to the reaction mixture which is kept stirring for 18 h at a temperature close to 20° C. It is then concentrated using a rotary evaporator under reduced pressure (5 kPa). The residue is triturated in diisopropyl ether then the solid is filtered, rinsed with diisopropyl ether and dried in the air. 194 mg of 6-{[(1S,2R)-1-benzyl-2-hydroxy-3-({1-[3-(trifluoromethyl)phenyl]cyclopropyl}amino)propyl]carbamoyl}-1,2,3,4-tetrahydropyrrolo[1,2-a]pyrazine-8-carboxylic acid hydrochloride (2:1) are obtained in the form of a yellow solid.

NMR: 1.19 (m, 1H); 1.28 (m, 1H); 1.58 (m, 2H); 2.71 (m, 2H); 2.94 (m, 1H); 3.05 (dd, J=3.0 and 14.0 Hz, 1H); 3.48 (m, 2H); 3.85 (m, 1H); 3.95 (m, 1H); 4.32 (m, 2H); 4.50 (s, 2H); 5.80 (broad m, 1H); 7.11 (m, 1H); 7.16 (s, 1H); 7.18 (m, 4H); 7.60 (t, J=7.5 Hz, 1H); 7.73 (d, J=7.5 Hz, 1H); 7.84 (d, J=7.5 Hz, 1H); 7.97 (s, 1H); 8.13 (d, J=9.0 Hz, 1H); from 9.26 to 10.0 (broad unresolved m, 4H); 12.4 (broad unresolved m, 1H).

LC-MS-DAD-ELSD: 557⁽⁺⁾=(M+H)⁽⁺⁾

MP: 196° C.

### EXAMPLE 4

### tert-Butyl 6-{[(1S,2R)-1-benzyl-2-hydroxy-3-({1-[3-(trifluoromethyl)phenyl]-cyclopropyl}amino)propyl]carbamoyl}-8-(diethylcarbamoyl)-3,4-dihydropyrrolo[1,2-a]pyrazine-2(1H)-carboxylate

0.6 g of 6-{[(1S,2R)-1-benzyl-2-hydroxy-3-({1-[3-(trifluoromethyl)phenyl]-cyclopropyl}amino)propyl]carbamoyl}-2-(tert-butoxycarbonyl)-1,2,3,4-tetrahydropyrrolo[1,2-a]pyrazine-8-carboxylic acid (prepared in example 2) and 0.094 cm³ of diethylamine are dissolved in 25 cm³ of dichloromethane at a temperature close to 20° C. 12 mg of hydroxybenzotriazole and 0.5 cm³ of N,N-diisopropylethylamine are added, followed by 210 mg of 1-(3-dimethylaminopropyl)-3-ethylcarbodiimide hydrochloride and 11 mg of 4-dimethylaminopyridine. The reaction mixture is kept stirring for 18 h at a temperature close to 20° C. It is washed successively with 10 cm³ of a 1M hydrochloric acid solution, with 20 cm³ of water, with 10 cm³ of a saturated aqueous solution of sodium hydrogen carbonate and with 15 cm³ of a saturated aqueous solution of sodium chloride. The organic phase is dried over a phase separator filter and concentrated using a rotary evaporator under reduced pressure (5 kPa). The foam obtained is purified by flash chromatography through a silica cartridge (column: 70 g); particle size: 15-40 μm; flow rate: 30 cm³/min; eluant: 20% cyclohexane/80% ethyl acetate). After concentrating the fractions under reduced pressure (5 kPa), 310 mg of tert-butyl 6-{[(1S,2R)-1-benzyl-2-hydroxy-3-({1-[3-(trifluoromethyl)-phenyl]cyclopropyl}amino)propyl]carbamoyl}-8-(diethylcarbamoyl)-3,4-dihydropyrrolo[1,2-a]pyrazine-2(1H)-carboxylate are obtained in the form of a white amorphous solid.

NMR: from 0.89 to 1.06 (m, 4H); 1.12 (t, J=7.0 Hz, 6H); 1.40 (s, 9H); 2.50 (masked m, 1H); 2.59 (m, 2H); 2.70 (dd, J=10.5 and 14.0 Hz, 1H); 3.03 (dd, J=3.5 and 14.0 Hz, 1H); 3.40 (q, J=7.0 Hz, 4H); 3.53 (m, 1H); 3.60 (m, 2H); 3.99 (m, 2H); 4.13 (m, 1H); 4.58 (m, 2H); 4.84 (d, J=6.0 Hz, 1H); 6.75 (s, 1H); 7.11 (m, 1H); 7.19 (m, 4H); from 7.42 to 7.51 (m, 2H); 7.54 (d, J=7.5 Hz, 1H); 7.63 (s, 1H); 7.88 (d, J=9.0 Hz, 1H).

LC-MS-DAD-ELSD: 710⁽⁻⁾=(M−H)⁽⁻⁾; 712⁽⁺⁾=(M+H)⁽⁺⁾

### EXAMPLE 5;

### 6-{(1S,2R)-1-benzyl-2-hydroxy-3-[1-(3-trifluoromethylphenyl)cyclopropylamino]propyl}-N-8,N-8-diethyl-1,2,3,4-tetrahydropyrrolo[1,2-a]pyrazine-6,8-dicarboxamide

200 mg of tert-butyl 6-{[(1S,2R)-1-benzyl-2-hydroxy-3-({1-[3-(trifluoromethyl)phenyl]cyclopropyl}amino)propyl]carbamoyl}-8-(diethylcarbamoyl)-3,4-dihydropyrrolo[1,2-a]pyrazine-2(1H)-carboxylate (prepared as in example 4) are dissolved in 5 cm³ of dichloromethane at a temperature close to 20° C. 0.70 cm³ of a 4M hydrochloric acid solution in dioxane are added. The reaction mixture is kept stirring for 18 h at a temperature close to 20° C. It is then concentrated using a rotary evaporator under reduced pressure (5 kPa). The residue is triturated in diisopropyl ether then the solid is filtered, rinsed with diisopropyl ether and dried in the air. 123 mg of 6-{(1S,2R)-1-benzyl-2-hydroxy-3-[1-(3-trifluoromethyl phenyl)cyclopropylamino]propyl}-N-8,N-8-diethyl-1,2,3,4-tetrahydropyrrolo[1,2-a]pyrazine-6,8-dicarboxamide hydrochloride (2:1) are obtained in the form of a yellow powder.

NMR: from 1.10 to 1.35 (m, 8H); 1.60 (m, 2H); from 2.61 to 2.81 (m, 2H); from 2.92 to 3.12 (m, 2H); from 3.25 to 3.58 (partially masked m, 6H); 4.90 (m, 2H); 4.22 (m, 1H); from 4.30 to 4.43 (m, 3H); 5.89 (broad unresolved m, 1H); 6.90 (s, 1H); 7.10 (t, J=7.5 Hz, 1H); from 7.13 to 7.24 (m, 4H); 7.61 (t, J=7.5 Hz, 1H); 7.77 (d, J=7.5 Hz, 1H); 7.87 (d, J=7.5 Hz, 1H); 8.00 (s, 1H); 8.24 (d, J=9.0 Hz, 1H); from 9.35 to 9.53 (broad unresolved m, 2H); 9.77 (broad unresolved m, 1H); 9.98 (broad unresolved m, 1H).

LC-MS-DAD-ELSD: 612⁽⁺⁾=(M+H)⁽⁺⁾

MP: 143° C.

### EXAMPLE 6

### 6-{(1S,2R)-1-Benzyl-2-hydroxy-3-[1-(3-trifluoromethylphenyl)cyclopropylamino]propyl}-N-8,N-8-diethyl-2-(methylsulfonyl)-1,2,3,4-tetrahydropyrrolo[1,2-a]pyrazine-6,8-dicarboxamide

### 6.1: 2-tert-Butyl 8-methyl 6-({(1S)-1-[(5R)-2-oxo-3-{1-[3-(trifluoromethyl)-phenyl]cyclopropyl}-1,3-oxazolidin-5-yl]-2-phenylethyl}carbamoyl)-3,4-dihydropyrrolo[1,2-a]pyrazine-2,8(1H)-dicarboxylate

Added to a solution of 2 g of 1.8: 2-tert-butyl 8-methyl 6-{[(1S,2R)-1-benzyl-2-hydroxy-3-({1-[3-(trifluoromethyl)phenyl]cyclopropyl}amino)propyl]-carbamoyl}-3,4-dihydropyrrolo[1,2-a]pyrazine-2,8(1H)-dicarboxylate (prepared in example 1.8) in 10 cm³ of dichloromethane cooled to a temperature close to 0° C. and under an inert atmosphere is 0.967 g of N,N′-carbonyldiimidazole then the mixture is stirred while leaving it to return to a temperature in the vicinity of 25° C. The stirring is continued for 48 h. 6 cm³ of dichloromethane and 15 cm³ of an aqueous solution of hydrochloric acid are added. The reaction mixture is extracted using two lots of 40 cm³ of dichloromethane. The organic phases are combined, dried then concentrated using a rotary evaporator under reduced pressure (5 kPa). The residual solid is purified by flash chromatography over silica (column: 200 g; particle size: 15-40 μm; flow rate: 90 cm³/min; eluant: 50% cyclohexane/50% ethyl acetate). The fractions containing the expected product are concentrated to dryness using a rotary evaporator under reduced pressure (5 kPa). 1.46 g of 2-tert-butyl 8-methyl 6-({(1S)-1-[(5R)-2-oxo-3-{1-[3-(trifluoromethyl)phenyl]cyclopropyl}-1,3-oxazolidin-5-yl]-2-phenylethyl}carbamoyl)-3,4-dihydropyrrolo[1,2-a]pyrazine-2,8(1H)-dicarboxylate are obtained in the form of a white solid.

MP: 106° C.

NMR 1.30-1.56 (partially masked m, 4H) 1.46 (s, 9H) 2.82 (dd, J=14.0, 10.7 Hz, 1H) 2.94 (d, J=14.0, 3.6 Hz, 1H) 3.60 (dd, J=9.0, 6.4 Hz, 1H) 3.68 (m, 2H) 3.75 (t, J=9.0 Hz, 1H) 3.79 (s, 3H) 4.13-4.29 (m, 2H) 4.38 (m, 1H) 4.62 (m, 1H) 4.79 (broad s, 2H) 7.19 (m, 1H) 7.27 (m, 5H) 7.48 (broad s, 1H) 7.52-7.66 (m, 3H, 8.18 (d, J=9.0 Hz, 1H).

LC-MS-DAD-ELSD: Tr (min)=2.44; MH+=697⁺; purity: 98%

### 6.2: Methyl 6-({(1S)-1-[(5R)-2-oxo-3-{1-[3-(trifluoromethyl)phenyl]cyclopropyl}-1,3-oxazolidin-5-yl]-2-phenylethyl}carbamoyl)-1,2,3,4-tetrahydropyrrolo[1,2-a]pyrazine-8-carboxylate

1.3 g of 2-tert-butyl 8-methyl 6-({(1S)-1-[(5R)-2-oxo-3-{1-[3-(trifluoromethyl)phenyl]cyclopropyl}-1,3-oxazolidin-5-yl]-2-phenylethyl}carbamoyl)-3,4-dihydropyrrolo[1,2-a]pyrazine-2,8(1H)-dicarboxylate are dissolved in 5 cm³ of dichloromethane under an inert atmosphere at a temperature close to 20° C., then 5 cm³ of a 4M hydrochloric acid solution in dioxane are added in 10 min. The reaction mixture is stirred for 15 h at a temperature in the vicinity of 20° C., then concentrated to dryness using a rotary evaporator under reduced pressure (5 kPa). The residual, pale yellow solid is taken up in 15 cm³ of diisopropyl ether and triturated. The solid is then filtered, rinsed with two lots of 10 cm³ of diisopropyl ether and dried in air in order to obtain 1.2 g of methyl 6-({(1S)-1-[(5R)-2-oxo-3-{1-[3-(trifluoromethyl)-phenyl]cyclopropyl}-1,3-oxazolidin-5-yl]-2-phenylethyl}carbamoyl)-1,2,3,4-tetrahydropyrrolo[1,2-a]pyrazine-8-carboxylate in the form of a white-cream dihydrochloride solid. This solid is used with no other purification in example 6.3.

NMR: 1.28-1.45 (m, 3H) 1.51 (m, 1H) 2.78 (dd, J=13.8, 10.5 Hz, 1H) 2.90 (dd, J=13.8, 3.8 Hz, 1H) 3.47 (m, 2H) 3.59 (m, 1H) 3.72 (t, J=8.9 Hz, 1H) 3.77 (s, 3H) 4.24-4.48 (m, 3H) 4.49 (s, 2H) 4.61 (m, 1H) 7.16 (m, 1H) 7.24 (m, 4H) 7.33 (s, 1H) 7.44 (broad s, 1H) 7.48-7.63 (m, 3H) 8.24 (d, =9.0 Hz, 1H) 9.52 (broad unresolved m, 2H)

LC-MS-DAD-ELSD: Tr (min)=3.26; MH+=597+; MH−=595−; purity: 98%

### 6.3: Methyl 2-(methylsulfonyl)-6-({(1S)-1-[(5R)-2-oxo-3-{1-[3-(trifluoromethyl)phenyl]cyclopropyl}-1,3-oxazolidin-5-yl]-2-phenylethyl}carbamoyl)-1,2,3,4-tetrahydropyrrolo[1,2-a]pyrazine-8-carboxylate

A solution of 0.132 cm³ of mesyl chloride in 2 cm³ of dichloromethane is added, under an inert atmosphere, and dropwise over 5 min, to a solution of 900 mg of methyl 6-({(1S)-1-[(5R)-2-oxo-3{1-[3-(trifluoromethyl)phenyl]cyclopropyl}-1,3-oxazolidin-5-yl]-2-phenylethyl}carbamoyl)-1,2,3,4-tetrahydropyrrolo[1,2-a]pyrazine-8-carboxylate, 0.5 cm³ of triethylamine and 30 cm³ of difluoromethane. The reaction mixture is stirred for 15 h at a temperature in the vicinity of 20° C., then 20 cm³ of water is added and the aqueous phase is brought to pH 2 by addition of a 1N aqueous hydrochloric acid solution. The organic phase is decanted and separated then washed with 20 cm³ of water. It is then dried over magnesium sulfate, then concentrated to dryness using a rotary evaporator under reduced pressure (5 kPa). The 940 mg of solid obtained are purified by flash chromatography over silica (column: 100 g; particle size: 15-40 μm; flow rate: 30 cm³/min; eluant: 50% cyclohexane/50% ethyl acetate). The fractions containing the expected product are concentrated to dryness using a rotary evaporator under reduced pressure (5 kPa). The 713 mg of colorless solid obtained are then taken up in 30 cm³ of tetrahydrofuran. 3 cm³ of a 4N hydrochloric acid solution in tetrahydrofuran are then added, and the mixture is left stirring at a temperature close to 20° C. for 1 h. The reaction mixture concentrated to dryness using a rotary evaporator under reduced pressure (5 kPa). 704 mg of a solid in the form of a white foam are obtained, which are triturated with 10 cm³ of diisopropyl ether. The solid is filtered, then dried in the air at a temperature in the vicinity of 20° C. 596 mg of methyl 2-(methylsulfonyl)-6-({(1S)-1-[(5R)-2-oxo-3-{1-[3-(trifluoromethyl)-phenyl]cyclopropyl}-1,3-oxazolidin-5-yl]-2-phenylethyl}carbamoyl)-1,2,3,4-tetrahydropyrrolo[1,2-a]pyrazine-8-carboxylate are obtained in the form of a white solid having a yellow sheen.

MP: 104° C.

NMR (400 MHz) 1.26-1.55 (m, 4H) 2.78 (dd, J=13.7, 10.3 Hz, 1H) 2.90 (dd, J=13.7, 3.9 Hz, 1H) 3.02 (s, 3H) 3.50-3.62 (m, 3H) 3.72 (t, J=8.8 Hz, 1H) 3.76 (s, 3H) 4.18-4.41 (m, 3H) 4.60 (m, 1H) 4.63 (s, 2H) 7.16 (m, 1H) 7.25 (m, 4H) 7.29 (s, 1H) 7.44 (broad s, 1H) 7.49-7.64 (m, 3H) 8.20 (d, J=9.3 Hz, 1H)

LC-MS-DAD-ELSD: Tr (min)=4.42; (M+H)⁽⁺⁾=675⁽⁺⁾; MH⁽⁻⁾=673⁽⁻⁾; purity: 98%

### 6.4: 2-(Methylsulfonyl)-6-({(1S)-1-[(5R)-3-{1-methyl-1-[3-(trifluoromethyl)-phenylethyl}-2-oxo-1,3-oxazolidin-5-yl]-2-phenylethyl}carbamoyl)-1,2,3,4-tetrahydropyrrolo[1,2-a]pyrazine-8-carboxylic acid

A mixture of 400 mg of methyl 2-(methylsulfonyl)-6-({(1S)-1-[(5R)-2-oxo-3-{1-[3-(trifluoromethyl)phenyl]cyclopropyl}-1,3-oxazolidin-5-yl]-2-phenylethyl}carbamoyl)-1,2,3,4-tetrahydropyrrolo[1,2-a]pyrazine-8-carboxylate, 10 cm³ of methanol and 2 cm³ of a 1N aqueous sodium hydroxide solution is heated for 3 h at a temperature close to reflux. The mixture is left to return to a temperature close to 20° C., then the solvents are evaporated using a rotary evaporator under reduced pressure (5 kPa). The residue of the evaporation is taken up in 10 cm³ of water and 10 cm³ of ethyl acetate. The organic phase is separated and the aqueous phase is acidified to pH ˜3-4 using a 1N aqueous hydrochloric acid solution. The aqueous phase is then extracted with 10 cm³ of ethyl acetate. The organic phase is separated, then washed successively with 10 cm³ of distilled water and 5 cm³ of a saturated solution of sodium chloride. The organic phase is dried over magnesium sulfate, then the solvents are evaporated to dryness using a rotary evaporator under reduced pressure (5 kPa). 330 mg of 2-(methyl sulfonyl)-6-({(1S)-1-[(5R)-3-{1-methyl-1-[3-(trifluoromethyl)phenyl]ethyl}-2-oxo-1,3-oxazolidin-5-yl]-2-phenylethyl}carbamoyl)-1,2,3,4-tetrahydropyrrolo[1,2-a]pyrazine-8-carboxylic acid are obtained in the form of a yellow-orange solid.

MP: 147° C.

NMR: 1.26-1.53 (m, 4H) 2.79 (dd, J=13.9, 10.3 Hz, 1H) 2.90 (dd, J=13.9, 3.9 Hz, 1H) 3.00 (s, 3H) 3.52 (t, J=5.6 Hz, 2H) 3.57 (dd, J=9.0, 6.1 Hz, 1H) 3.71 (t, J=9.0 Hz, 1H) 4.15-4.40 (m, 3H) 4.58 (m, 1H) 4.63 (s, 2H) 7.16 (m, 1H) 7.21 (s, 1H) 7.24 (m, 4H) 7.45 (broad s, 1H) 7.49-7.64 (m, 3H) 8.13 (d, J=8.8 Hz, 1H) 12.25 (broad unresolved m, 1H)

LC-MS-DAD-ELSD: Tr (min)=4.14; (M+H)⁽⁺⁾=661⁽⁺⁾; purity: 98%

### 6.5: N-8,N-Diethyl-2-(methylsulfonyl)-N-6-{(1S)-1-[(5R)-2-oxo-3-{1-[3-(trifluoromethyl)phenyl]cyclopropyl}-1,3-oxazolidin-5-yl]-2-phenylethyl}-1,2,3,4-tetrahydropyrrolo[1,2-a]pyrazine-6,8-dicarboxamide

Introduced into a three-necked flask are successively 5 cm³ of dimethylformamide, 0.170 cm³ of diethylamine and 0.05 cm³ of diisopropylethylamine. A solution of 116 mg of 1-[bis(dimethylamino)methylene]-1H-1,2,3-triazolo[4,5-b]pyridinium-3-oxide hexafluorophosphate (HATU) and 125 mg of 2-(methylsulfonyl)-6-({(1S)-1-[(5R)-3-{1-methyl-1-[3-(trifluoromethyl)-phenyl]ethyl}-2-oxo-1,3-oxazolidin-5-yl]-2-phenylethyl}carbamoyl)-1,2,3,4-tetrahydropyrrolo[1,2-a]pyrazine-8-carboxylic acid in 10 cm³ of dimethylformamide is added dropwise over 10 minutes. The reaction mixture is left stirring for 48 h at a temperature in the vicinity of 20° C. 10 cm³ of ethyl acetate and 15 cm³ of a 0.1N aqueous solution of hydrochloric acid are added to the reaction medium. The organic phase is separated, then washed successively with 10 cm³ of water and 5 cm³ of a saturated aqueous solution of sodium chloride. It is then dried over sodium sulfate and concentrated using a rotary evaporator under reduced pressure (5 kPa). The residual solid obtained is purified by flash chromatography over silica (column: 25 g; particle size: 15-40 μm; flow rate: 10 cm³/min; eluant: 100% ethyl acetate). The fractions containing the expected product are concentrated to dryness using a rotary evaporator under reduced pressure (5 kPa). 100 mg are obtained of a solid which is taken up and triturated with 3 cm³ of diisopropyl ether results in 93 mg of N-8,N-8-diethyl-2-(methylsulfonyl)-N-6-{(1S)-1-[(5R)-2-oxo-3-{1-[3-(trifluoromethyl)phenyl]-cyclopropyl}-1,3-oxazolidin-5-yl]-2-phenylethyl}-1,2,3,4-tetrahydropyrrolo-[1,2-a]pyrazine-6,8-dicarboxamide in the form of a white solid having a cream-colored sheen.

MP: 109° C.

NMR: 1.16 (t, J=7.1 Hz, 6H) 1.28-1.54 (m, 4H) 2.79 (dd, J=13.7, 10.3 Hz, 1H) 2.92 (dd, J=13.7, 3.9 Hz, 1H) 2.97 (s, 3H) 3.36-3.62 (m, 7H) 3.75 (t, J=8.8 Hz, 1H) 4.17-4.32 (m, 3H) 4.46 (m, 2H) 4.60 (m, 1H) 6.87 (s, 1H) 7.16 (t, J=7.5 Hz, 1H) 7.19-7.30 (m, 4H) 7.48 (broad s, 1H) 7.50-7.66 (m, 3H) 8.09 (d, J=9.3 Hz, 1H)

LC-MS-DAD-ELSD: Tr (min)=5.08; (M+H)⁽⁺⁾=716⁽⁺⁾; MH⁽⁻⁾=714⁽⁻⁾; purity: 98%

### 6.6: 6-{(1S,2R)-1-Benzyl-2-hydroxy-3-[1-(3-trifluoromethylphenyl)cyclopropylamino]propyl}-N-8,N-8-diethyl-2-(methylsulfonyl)-1,2,3,4-tetrahydropyrrolo[1,2-a]pyrazine-6,8-dicarboxamide

Added to a solution of 60 mg of N-8,N-8-diethyl-2-(methylsulfonyl)-N-6-{(1S)-1-[(5R)-2-oxo-3-{1-[3-(trifluoromethyl)phenyl]cyclopropyl}-1,3-oxazolidin-5-yl]-2-phenylethyl}-1,2,3,4-tetrahydropyrrolo[1,2-a]pyrazine-6,8-dicarboxamide in 2 cm³ of isopropanol and 0.2 cm³ of methanol is 1 cm³ of a 30% aqueous solution of sodium hydroxide. The reaction mixture is heated for 2 h at a temperature close to reflux. The mixture is then left to return to a temperature close to 20° C., then the solvents are evaporated using a rotary evaporator under reduced pressure (5 kPa). The residue of the evaporation is taken up with 5 cm³ of water and 5 cm³ of ethyl acetate.

The organic phase is separated and the aqueous phase is acidified to pH ˜4-5 using a 1N aqueous solution of hydrochloric acid. The aqueous phase is then extracted with 10 cm³ of ethyl acetate. The organic phase is separated, then washed successively with two lots of 5 cm³ of distilled water, then dried over magnesium sulfate. The organic phase is concentrated to dryness using a rotary evaporator under reduced pressure (5 kPa). The residual solid obtained (38 mg) is purified by flash chromatography over silica (column: 15 g; particle size: 15-40 μm; flow rate: 10 cm³/min; eluant: 100% ethyl acetate). The fractions containing the expected product are concentrated to dryness using a rotary evaporator under reduced pressure (5 kPa). 6 mg of 6-{(1S,2R)-1-benzyl-2-hydroxy-3-[1-(3-trifluoromethylphenyl)cyclopropylamino]propyl}-N-8,N-8-diethyl-2-(methylsulfonyl)-1,2,3,4-tetrahydropyrrolo[1,2-a]pyrazine-6,8-dicarboxamide are obtained in the form of a white solid.

NMR: from 0.89 to 1.06 (m, 4H); 1.14 (t, J=7.0 Hz, 6H); from 2.42 to 2.77 (partially masked m, 3H); 2.98 (s, 3H); 3.02 (dd, J=4.0 and 13.0 Hz, 1H); from 3.23 to 3.59 (partially masked m, 7H); 3.98 (m, 1H); 4.10 (m, 1H); 4.23 (m, 1H); 4.45 (m, 2H); 4.87 (d, J=5.0 Hz, 1H); 6.80 (s, 1H); from 7.07 to 7.23 (m, 5H); from 7.42 to 7.58 (m, 3H); 7.64 (broad s, 1H); 7.91 (d, J=8.5 Hz, 1H).

### EXAMPLE 7

### 7.1: Base

### 6-[(1S,2R)-1-(3,5-Difluorobenzyl)-2-hydroxy-3-({1-[3-(trifluoromethyl)-phenyl]cyclopropyl}amino)propyl]-1-oxo-2-(1-propylbutyl)-1,2,3,4-tetrahydropyrrolo[1,2-a]pyrazine-6-carboxamide

### 7.1.1: Ethyl 1-(2-bromoethyl)-1H-pyrrole-2-carboxylate

5 g of ethyl pyrrole-2-carboxylate, 15.5 cm³ of 1,2-dibromoethane, 21.5 cm³ of sodium hydroxide and 11.58 g of tetrabutylammonium bromide are stirred under an inert atmosphere for 20 h at a temperature close to 20° C. 100 cm³ of dichloromethane and 50 cm³ of water are added to the reaction medium. The aqueous phase is extracted with three lots of 20 cm³ of dichloromethane. The organic phases are combined then washed with 50 cm³ of a saturated aqueous solution of sodium chloride, dried over magnesium sulfate and concentrated using a rotary evaporator under reduced pressure (5 kPa). The 29.5 g of clear oil attained are purified by flash chromatography over silica (column: 400 g; particle size: 15-40 μm; eluant: 100% dichloromethane). After concentrating the fractions under reduced pressure, 6.78 g of ethyl 1-(2-bromoethyl)-1H-pyrrole-2-carboxylate are obtained.

LC-MS-DAD-ELSD: 246⁽⁺⁾⁷⁹Br=(M+H)⁽⁺⁾

NMR: 1.27 (t, J=7.1 Hz, 3H) 3.74 (t, J=6.5 Hz, 2H) 4.21 (q, J=7.1 Hz, 2H) 4.65 (t, J=6.5 Hz, 2H) 6.13 (dd, J=3.9, 2.6 Hz, 1H) 6.88 (dd, J=3.9, 1.8 Hz, 1H) 7.20 (dd, J=2.6, 1.8 Hz, 1H)

### 7.1.2: Ethyl 1-{2-[(1-propylbutyl)amino]ethyl}-1H-pyrrole-2-carboxylate

6.76 g of ethyl 1-(2-bromoethyl)-1H-pyrrole-2-carboxylate, 11.08 g of 4-heptylamine, 0.46 g of potassium iodide and 135 cm³ of acetonitrile are stirred at 70° C. under an inert atmosphere for 20 h. The reaction mixture is brought to 30° C. in order to be concentrated using a rotary evaporator under reduced pressure (5 kPa). 200 cm³ of dichloromethane and 100 cm³ of water are then added to the concentration residue. The phases are stirred for 5 min, then are separated. The aqueous phase is extracted with three lots of 50 cm³ of dichloromethane. The organic phases are combined, dried over magnesium sulfate and concentrated using a rotary evaporator under reduced pressure (5 kPa). The 10.8 g of amber-colored oil obtained are purified by flash chromatography over silica (column: 450 g; particle size: 20-40 μm, spherical; eluant: 70% cyclohexane/30% ethyl acetate to 50% cyclohexane/50% ethyl acetate gradient). After concentrating the fractions under reduced pressure, 4.31 g of ethyl 1-{2-[(1-propylbutyl)-amino]ethyl}-1H-pyrrole-2-carboxylate are obtained.

MS-EI: 280⁽⁺⁾=M^{*}⁽⁺⁾

NMR: 0.82 (m, 6H) 0.93-1.32 (m, 11H) 2.36 (m, 1H) 2.77 (t, J=6.6 Hz, 2H) 4.19 (q, J=7.1 Hz, 2H) 4.29 (t, J=6.6 Hz, 2H) 6.07 (dd, J=4.0, 2.5 Hz, 1H) 6.83 (dd, J=4.0, 1.8 Hz, 1H) 7.12 (dd, J=2.5, 1.8 Hz, 1H)

### 7.1.3: 2-(1-Propylbutyl)-3,4-dihydropyrrolo[1,2-a]pyrazin-1(2H)-one

4.3 g of ethyl 1-{2-[(1-propylbutyl)amino]ethyl}-1H-pyrrole-2-carboxylate are dissolved under an inert atmosphere, in 130 cm³ of toluene at a temperature close to 20° C. 23 cm³ of a 2M toluene solution of trimethylaluminum are poured over the reaction mixture over 5 min. The reaction mixture is heated, with stirring, for 20 h at 110° C., then 72 h at 20° C. It is then poured over an ice/water/ethyl acetate mixture. The suspension obtained is filtered over a Celite 545 pellet. The aqueous phase is extracted with three lots of 75 cm³ of ethyl acetate. The organic phases are combined, then washed with 50 cm³ of a saturated aqueous solution of sodium chloride, dried over magnesium sulfate and concentrated using a rotary evaporator under reduced pressure (5 kPa). The 4.7 g of brown oil obtained are purified by flash chromatography over silica (column: 200 g; particle size: 15-40 μm; eluant: 100% dichloromethane to 80% dichloromethane/20% ethyl acetate gradient). After concentrating the fractions under reduced pressure, 1.56 g of 2-(1-propylbutyl)-3,4-dihydropyrrolo[1,2-a]pyrazin-1(2H)-one are obtained.

MS-EI: 234⁽⁺⁾=M⁽⁺⁾

NMR: 0.86 (t, J=7.3 Hz, 6H) 1.12-1.29 (m, 4H) 1.39 (m, 2H) 1.47 (m, 2H) 3.43 (m, 2H) 4.10 (m, 2H) 4.58 (m, 1H) 6.12 (dd, J=3.8, 2.5 Hz, 1H) 6.59 (dd, J=3.8, 1.5 Hz, 1H) 6.94 (dd, J=2.5, 1.5 Hz, 1H)

### 7.1.4: 6-Bromo-2-(1-propylbutyl)-3,4-dihydropyrrolo[1,2-a]pyrazin-1(2H)-one

1.53 g of 2-(1-propylbutyl)-3,4-dihydropyrrolo[1,2-a]pyrazin-1(2H)-one and 1.16 g of N-bromosuccinimide are dissolved in 153 cm³ of carbon tetrachloride at a temperature close to 20° C. Stirring is continued for 2 h 30 min. The reaction medium is concentrated using a rotary evaporator under reduced pressure (5 kPa). The solid residue obtained is purified by flash chromatography over silica (column: 200 g; particle size: 15-40 μm; eluant: 100% dichloromethane to 90% dichloromethane/10% ethyl acetate gradient). After concentrating the fractions under reduced pressure, 1.54 g of 6-bromo-2-(1-propylbutyl)-3,4-dihydropyrrolo[1,2-a]pyrazin-1(2H)-one are obtained.

NMR: 0.86 (t, J=7.5 Hz, 6H); from 1.10 to 1.29 (m, 4H): from 1.32 to 1.54 (m, 4H); 3.48 (m, 2H); 4.02 (m, 2H); 4.56 (m, 1H); 6.30 (d, J=4.0 Hz, 1H); 6.68 (d, J=4.0 Hz, 1H).

MS-EI: M⁽⁺⁾.=312⁽⁺⁾.

### 7.1.5: 1-Oxo-2-(1-propylbutyl)-1,2,3,4-tetrahydropyrrolo[1,2-a]pyrazine-6-carboxylic acid

Introduced successively, at a temperature close to 20° C., into a three-necked flask that is stirred and purged using carbon monoxide are 800 mg of 6-bromo-2-(1-propylbutyl)-3,4-dihydropyrrolo[1,2-a]pyrazin-1(2H)-one, 27 cm³ of dimethylformamide, 1.3 cm³ of water, 0.953 g of potassium acetate, 85 mg of potassium iodide, 230 mg of palladium acetate and 535 mg of triphenylphosphine. The reaction mixture is subjected to a carbon monoxide bubbling, then is heated at 100° C. for 6 h. The reaction mixture is kept at 100° C. for 20 h, then cooled to 25° C. in order to be filtered over a 45 μm Millipore membrane. The filtrate is concentrated using a rotary evaporator under reduced pressure (5 kPa). The oily residue obtained is taken up in 15 cm³ of a water/ice mixture and 20 cm³ of ethyl acetate. The pH is alkalinized with 5M sodium hydroxide (pH>10). After decantation of the filtrate, the aqueous phase is washed with three lots of 20 cm³ of ethyl acetate. It is then acidified, with stirring, with a 5M hydrochloric acid solution (pH=1), then extracted with three lots of 20 cm³ of ethyl acetate. The organic phases are combined, then washed with 10 cm³ of a saturated aqueous solution of sodium chloride, dried over magnesium sulfate and concentrated using a rotary evaporator under reduced pressure (5 kPa). The 1.2 g of orange oil obtained are purified by flash chromatography over silica (column: 90 g; particle size: 15-40 μm; eluant: 100% dichloromethane to 95% dichloromethane/5′)/0 methanol gradient). After concentrating the fractions under reduced pressure, 0.69 g of 1-oxo-2-(1-propylbutyl)-1,2,3,4-tetrahydropyrrolo[1,2-a]pyrazine-6-carboxylic acid is obtained.

MS-EI: 278⁽⁺⁾=M⁽⁺⁾; 261⁽⁺⁾=M⁽⁺⁾—OH

NMR: 86 (t, J=7.2 Hz, 6H) 1.12-1.57 (m, 8H) 3.49 (m, 2H) 4.49 (m, 2H) 4.57 (m, 1H) 6.66 (d, J=4.0 Hz, 1H) 6.81 (d, J=4.0 Hz, 1H) 12.78 (broad unresolved m, 1H)

### 7.1.6: tert-Butyl[(1S,2R)-1-(3,5-difluorobenzyl)-2-hydroxy-3-({1-[3-(trifluoromethyl)phenyl]cyclopropyl}amino)propyl]carbamate

Added to a solution of 0.794 g of 1-(3-trifluoromethyl)phenylcyclopropylamine hydrochloride in 20 cm³ of water are 4 cm³ of 1M sodium hydroxide at a temperature close to 20° C. This solution is stirred for 15 min. 40 cm³ of dichloromethane are added and the mixture is stirred for 5 min. The aqueous phase is extracted with 30 cm³ of dichloromethane. The organic phases are combined, dried over magnesium sulfate and concentrated using a rotary evaporator under reduced pressure (5 kPa). 700 mg of 1-(3-trifluoromethyl)phenylcyclopropylamine are obtained, which are dissolved in 12 cm³ of dichloromethane. 1 g of tert-butyl[(1S)-2-(3,5-difluorophenyl)-1-oxiran-2-ylethyl]carbamate and 0.329 g of scandium triflate are added to the solution. The light orange-colored suspension is kept stirring at ambient temperature for 20 h. The reaction mixture is concentrated using a rotary evaporator under reduced pressure (5 kPa). The crude product obtained is purified by flash chromatography over silica (column: 200 g; particle size: 15-40 μm; flow rate: 50 cm³/min; eluant: 70% cyclohexane/30% ethyl acetate). After concentrating the fractions under reduced pressure, 0.87 g of tert-butyl[(1S,2R)-1-(3,5-difluorobenzyl)-2-hydroxy-3-({1-[3-(trifluoromethyl)phenyl]cyclopropyl}amino)propyl]carbamate is obtained in the form of a white solid.

LC-MS-DAD-ELSD: 501⁽⁺⁾=(M+H)⁽⁺⁾; 445⁽⁺⁾=(M+H)⁽⁺⁾-tBu+H

NMR: 0.90-1.04 (m, 4H) 1.22 (s, 9H) 2.40 (m, 1H) 2.46-2.62 (partially masked m, 2H) 2.99 (dd, J=14.1, 3.2 Hz, 1H) 3.36 (m, 1H) 3.52 (m, 1H) 4.80 (d, J=5.9 Hz, 1H) 6.66 (d, J=9.3 Hz, 1H) 6.86 (m, 2H) 6.99 (tt, J=9.2, 2.4 Hz, 1H) 7.48-7.61 (m, 3H) 7.65 (broad s, 1H)

### 7.1.7: (2R,3S)-3-Amino-4-(3,5-difluorophenyl)-1-({1-[3-(trifluoromethyl)-phenyl]cyclopropyl}amino)butan-2-ol hydrochloride (1:1)

At a temperature of 20° C., 870 mg of tert-butyl[(1S,2R)-1-(3,5-difluorobenzyl)-2-hydroxy-3-({1-[3-(trifluoromethyl)phenyl]cyclopropyl}-amino)propyl]carbamate are dissolved in 20 cm³ of dichloromethane. 17.38 cm³ of a 1M hydrochloric acid solution in ethyl ether are added to the reaction mixture which is kept stirring for 20 h at ambient temperature, then concentrated using a rotary evaporator under reduced pressure (5 kPa). 0.82 g of (2R,3S)-3-amino-4-(3,5-difluorophenyl)-1-({1-[3-(trifluoromethyl)phenyl]cyclopropyl}amino)butan-2-ol hydrochloride (1:1) is obtained in the form of a white solid.

LC-MS-DAD-ELSD: 401⁽⁺⁾=(M+H)⁽⁺⁾

NMR: For this batch, all the signals are broad with: 1.13-1.37 (m, 2H) 1.53-1.71 (m, 2H) 2.71 (m, 1H) 2.84 (dd, J=14.3, 8.2 Hz, 1H) 2.93 (dd, J=14.3, 5.9 Hz, 1H) 3.09 (m, 1H) 3.57 (m, 1H) 4.18 (m, 1H) 6.19 (m, 1H) 7.01-7.10 (m, 3H) 7.65 (t, J=7.8 Hz, 1H) 7.77 (d, J=7.8 Hz, 1H) 7.87 (d, J=7.8 Hz, 1H) 7.98 (s, 1H) 8.19 (s, 3H) 9.77 (m, 1H) 10.23 (m, 1H)

### 7.1.8: N-[(1S,2R)-1-(3,5-Difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide

Poured into a suspension of 80 mg of 1-oxo-2-(1-propylbutyl)-1,2,3,4-tetrahydropyrrolo[1,2-a]pyrazine-6-carboxylic acid, 136 mg of (2R,3S)-3-amino-4-(3,5-difluorophenyl)-1-({1-[3-(trifluoromethyl)phenyl]cyclopropyl}amino)-butan-2-ol hydrochloride (1:1), 6 mg of hydroxybenzotriazole and 69 mg of 1-(3-dimethylaminopropyl)-3-ethylcarbodiimide hydrochloride in 10 cm³ of dichloromethane is 0.246 cm³ of N,N-diisopropylethylamine at a temperature close to 20° C. The solution is kept stirring for 20 h at this temperature. 30 cm³ of dichloromethane and 15 cm³ of water are added to the reaction medium. The organic phase is washed with 10 cm³ of a saturated aqueous solution of sodium chloride, dried over sodium sulfate and concentrated using a rotary evaporator under reduced pressure (5 kPa). The 310 mg of brown oil obtained are purified by flash chromatography over silica (column: 35 g; particle size: 20-40 μm, spherical; eluant: 100% ethyl acetate). After concentrating the fractions under reduced pressure, 116 mg of N-[(1S,2R)-1-(3,5-difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide are obtained.

LC-MS-DAD-ELSD: 661⁽⁺⁾=(M+H)⁽⁺⁾

NMR: 0.86 (t, J=7.1 Hz, 3H) 0.87 (t, J=7.1 Hz, 3H) 0.92-1.54 (m, 12H) 2.44-2.72 (partially masked m, 3H) 2.76 (dd, J=13.9, 10.6 Hz, 1H) 3.06 (dd, J=13.9, 3.4 Hz, 1H) 3.30-3.42 (masked m, 2H) 3.55 (m, 1H) 4.08 (m, 1H) 4.26 (m, 1H) 4.35 (m, 1H) 4.56 (m, 1H) 4.98 (d, J=6.0 Hz, 1H) 6.62 (d, J=4.1 Hz, 1H) 6.77 (d, J=4.1 Hz, 1H) 6.94 (m, 2H) 6.99 (tt, J=9.2, 2.0 Hz, 1H) 7.43-7.51 (m, 2H) 7.55 (broad d, J=7.6 Hz, 1H) 7.66 (broad s, 1H) 8.06 (d, J=9.0 Hz, 1H)

### 7.2: Salt

### N-[(1S,2R)-1-(3,5-Difluorobenzyl)-2-hydroxy-3-({1-[3-(trifluoromethyl)-phenyl]cyclopropyl}amino)propyl]-1-oxo-2-(1-propylbutyl)-1,2,3,4-tetrahydropyrrolo[1,2-a]pyrazine-6-carboxamide hydrochloride (1:1)

At a temperature close to 20° C., 112 mg of N-[(1S,2R)-1-(3,5-difluorobenzyl)-2-hydroxy-3-({1-[3-(trifluoromethyl)phenyl]cyclopropyl}amino)-propyl]-1-oxo-2-(1-propylbutyl)-1,2,3,4-tetrahydropyrrolo[1,2-a]pyrazine-6-carboxamide are dissolved in 1.5 cm³ of ethyl ether. 0.43 cm³ of a 2M hydrochloric acid solution in ethyl ether is added, while stirring under argon, at a temperature of 5° C. The reaction mixture precipitates. The stirring is continued for 20 min, then is stopped in order to remove the supernatant. 3 cm³ of ethyl ether are again added, then removed. This operation is carried out twice. The last suspension is concentrated under reduced pressure (5 kPa). 117 mg of N-[(1S,2R)-1-(3,5-difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide hydrochloride (1:1) are obtained in the form of a white solid.

MP: 183° C.

NMR: 0.85 (t, J=7.5 Hz, 3H); 0.87 (t, J=7.5 Hz, 3H); from 1.12 to 1.65 (m, 12H); 2.72 (m, 1H); 2.79 (m, 1H); 2.98 (m, 1H); 3.08 (dd, J=3.0 and 14.0 Hz, 1H); 3.40 (partially masked m, 2H); 3.84 (m, 1H); 3.98 (m, 1H); 4.20 (m, 1H); 4.35 (m, 1H); 4.56 (m, 1H); 5.83 (very broad unresolved m, 1H); 6.62 (d, J=4.0 Hz, 1H); 6.74 (d, J=4.0 Hz, 1H); from 6.90 to 7.02 (m, 3H); 7.59 (t, J=7.5 Hz, 1H); 7.71 (d, J=7.5 Hz, 1H); 7.88 (d, J=7.5 Hz, 1H); 7.97 (s, 1H); 8.16 (d, J=9.0 Hz, 1H); 9.35 (broad m, 1H); 9.62 (broad m, 1H).

LC-MS-DAD-ELSD: 659⁽⁻⁾=(M−H)⁽⁻⁾; 661⁽⁺⁾=(M+H)⁽⁺⁾

### EXAMPLE 8

### 8.1: Base

### N-[(1S,2R)-1-Benzyl-2-hydroxy-3-({1-[3-(trifluoromethyl)phenyl]cyclopropyl}amino)propyl]-1-oxo-2-(1-propyl butyl)-1,2,3,4-tetrahydropyrrolo[1,2-a]pyrazine-6-carboxamide

Poured into a suspension of 50 mg of 1-oxo-2-(1-propylbutyl)-1,2,3,4-tetrahydropyrrolo[1,2-a]pyrazine-6-carboxylic acid, 85 mg of (2R,3S)-3-amino-4-phenyl-1-({1-[3-(trifluoromethyl)phenyl]cyclopropyl}amino)butan-2-ol hydrochloride, 3.6 mg of hydroxybenzotriazole and 43 mg of 1-(3-dimethylaminopropyl)-3-ethylcarbodiimide hydrochloride in 8 cm³ of dichloromethane is 0.185 cm³ of N,N-diisopropylethylamine at a temperature close to 20° C. The solution obtained is kept stirring for 20 h. 20 cm³ of dichloromethane and 10 cm³ of water are added to the reaction medium. The organic phase is washed with 10 cm³ of a saturated aqueous solution of sodium chloride, dried over sodium sulfate and concentrated using a rotary evaporator under reduced pressure (5 kPa). The 170 mg of oil obtained are purified by flash chromatography over silica (column: 15 g; particle size: 20-40 μm, spherical; eluant: 100% dichloromethane to 95% dichloromethane/5% methanol gradient). After concentrating the fractions under reduced pressure, 90 mg of N-[(1S,2R)-1-benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide are obtained.

LC-MS-DAD-ELSD: 623⁽⁻⁾=(M−H)“; 669⁽⁻⁾=(M+formic acid-H)⁽⁻⁾; 625⁽⁺⁾=(M+H)⁽⁺⁾

NMR: 0.84 (t, J=7.3 Hz, 3H) 0.86 (t, J=7.3 Hz, 3H) 0.90-1.54 (m, 12H) 2.45-2.60 (partially masked m, 2H) 2.71 (dd, J=13.9, 10.5 Hz, 1H) 3.04 (dd, J=13.9, 3.6 Hz, 1H) 3.17-3.47 (partially masked m, 3H) 3.54 (m, 1H) 4.03 (m, 1H) 4.23 (m, 1H) 4.33 (m, 1H) 4.54 (m, 1H) 4.86 (d, J=6.1 Hz, 1H) 6.59 (d, J=4.1 Hz, 1H) 6.73 (d, J=4.1 Hz, 1H) 7.12 (m, 1H) 7.21 (m, 4H) 7.42-7.59 (m, 3H) 7.64 (broad s, 1H) 8.01 (d, J=9.0 Hz, 1H).

### 8.2 Salt

### N-[(1S,2R)-1-Benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide hydrochloride (1:1)

At a temperature close to 20° C., 85 mg of N-[(1S,2R)-1-benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide are dissolved in 1 cm³ of ethyl ether. 0.4 cm³ of a 2M hydrochloric acid solution in ethyl ether are added while stirring under argon, at a temperature of 5° C. The reaction mixture precipitates. The stirring is continued for 15 min, then is stopped in order to remove the supernatant. 3 cm³ of ethyl ether are again added, then removed. This operation is carried out three times. The last suspension is then concentrated under reduced pressure (5 kPa). 87 mg of N-[(1S,2R)-1-benzyl-2-hydroxy-3-({1-[3-(trifluoromethyl)phenyl]-cyclopropyl}amino)propyl]-1-oxo-2-(1-propylbutyl)-1,2,3,4-tetrahydropyrrolo[1,2-a]pyrazine-6-carboxamide hydrochloride (1:1) are obtained in the form of a white solid.

MP: 176° C.

NMR: 0.84 (t, J=7.5 Hz, 3H); 0.87 (t, J=7.5 Hz, 3H); from 1.11 to 1.62 (m, 12H); 2.74 (m, 2H); 2.95 (m, 1H); 3.09 (dd, J=3.0 and 14.0 Hz, 1H); 3.38 (partially masked m, 2H); 3.83 (m, 1H); 3.94 (m, 1H); 4.17 (m, 1H); 4.34 (m, 1H); 4.55 (m, 1H); 5.82 (very broad unresolved m, 1H); 6.61 (d, J=4.0 Hz, 1H); 6.72 (d, J=4.0 Hz, 1H); 7.11 (m, 1H); 7.20 (m, 4H); 7.58 (t, J=7.5 Hz, 1H); 7.71 (d, J=7.5 Hz, 1H); 7.87 (d, J=7.5 Hz, 1H); 7.96 (s, 1H); 8.14 (d, J=9.0 Hz, 1H); 9.33 (broad m, 1H); 9.61 (broad m, 1H).

LC-MS-DAD-ELSD: 623⁽⁻⁾=(M−H)⁽⁻⁾; 625⁽⁺⁾=(M+H)⁽⁺⁾

### EXAMPLE 9

### 9.1: Base

### N-[(1S,2R)-1-Benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide

### 9.1.1: tert-Butyl[(1S,2R)-1-benzyl-2-hydroxy-3-{[3-(trifluoromethyl)benzyl]-amino}propyl]carbamate

Poured, over 20 min, into a suspension, under argon, of 10 g of tert-butyl [S—(R,R)]-(−)-(1-oxiranyl-2-phenylethyl)carbamate in 100 cm³ of 2-propanol is a solution of 6.5 cm³ of 3-(trifluoromethyl)benzylamine in 20 cm³ of 2-propanol at a temperature close to 20° C. The reaction mixture is heated at a temperature of 65° C. for 20 h. It is then cooled in order to be concentrated using a rotary evaporator under reduced pressure (5 kPa). The 20 g of colorless oil obtained are purified by flash chromatography over silica (column: 400 g; particle size: 15-40 μm; flow rate: 20 cm³/min; eluant: 100% dichloromethane to 95% dichloromethane/5% methanol gradient over 140 min). After concentrating the fractions under reduced pressure, 11 g of tert-butyl[(1S,2R)-1-benzyl-2-hydroxy-3-{[3-(trifluoromethyl)benzyl]amino}propyl]carbamate are obtained in the form of a white solid.

LC-MS-DAD-ELSD: 439⁽⁺⁾=(M+H)⁽⁺⁾; 483⁽⁻⁾=(M+formic acid-H)⁽⁻⁾

NMR: For this batch, all the signals are broad with: 1.22 (s, 9H) 2.25 (m, 1H) 2.38-2.65 (partially masked m, 3H) 2.99 (m, 1H) 3.51-3.65 (m, 2H) 3.80 (m, 2H) 4.83 (d, J=6.3 Hz, 1H) 6.61 (d, J=9.1 Hz, 1H) 6.99-7.30 (m, 5H) 7.48-7.80 (m, 4H).

### 9.1.2: (2R,3S)-3-Amino-4-phenyl-1-{[3-(trifluoromethyl)benzyl]amino}butan-2-ol hydrochloride (1:1)

11.2 g of tert-butyl[(1S,2R)-1-benzyl-2-hydroxy-3-{[3-(trifluoromethyl)-benzyl]amino}propyl]carbamate are dissolved in 111 cm³ of dichloromethane at a temperature close to 20° C. 63 cm³ of a 4M hydrochloric acid solution in dioxane are added at a temperature of 0° C. The reaction mixture precipitates. It is kept stirring for 2 h at 20° C. The precipitate is filtered, washed with four lots of 30 cm³ of dichloromethane and three lots of 30 cm³ of diisopropyl ether. The white solid obtained is dried in a dessicator for two days. 10.5 g of (2R,3S)-3-amino-4-phenyl-1-{[3-(trifluoromethyl)benzyl]amino}butan-2-ol are obtained.

LC-MS-DAD-ELSD: 339⁽⁺⁾=(M+H)⁽⁺⁾

NMR: 2.79-2.96 (m, 3H) 3.13 (m, 1H) 3.46-3.59 (partially masked m, 1H) 4.16-4.30 (m, 3H) 6.30 (broad m, 1H) 7.21-7.36 (m, 5H) 7.67 (t, J=7.8 Hz, 1H) 7.79 (d, J=7.8 Hz, 1H) 7.87 (d, J=7.8 Hz, 1H) 8.00 (s, 1H) 8.21 (broad s, 3H) 9.41 (broad m, 1H) 9.72 (broad m, 1H).

### 9.1.3: (2R,3S)-3-Amino-4-phenyl-1-{[3-(trifluoromethyl)benzyl]amino}butan-2-ol

Added to a solution of 0.6 g of (2R,3S)-3-amino-4-phenyl-1-{[3-(trifluoromethyl)benzyl]amino}butan-2-ol hydrochloride (1:1) in 30 cm³ of dichloromethane are 10 cm³ of water and 2.9 cm³ of 1M sodium hydroxide at a temperature close to 20° C. The reaction mixture is kept stirring for 1 h. The organic phase is dried over magnesium sulfate, then filtered. The filtrate is evaporated under reduced pressure (5 kPa). 360 mg of (2R,3S)-3-amino-4-phenyl-1-{[3-(trifluoromethyl)benzyl]amino}butan-2-ol are obtained in the form of a colorless oil which crystallizes.

NMR: 1.67 (broad m, 3H) 2.35 (dd, J=12.5, 8.1 Hz, 1H) 2.56 (dd, J=11.9, 7.4 Hz, 1H) 2.68 (dd, J=12.0, 3.7 Hz, 1H) 2.76-2.89 (m, 2H) 3.38 (partially masked m, 1H) 3.80 (s, 2H) 4.65 (broad s, 1H) 7.11-7.22 (m, 3H) 7.24-7.30 (m, 2H) 7.52-7.61 (m, 2H) 7.64 (broad d, J=7.6 Hz, 1H) 7.70 (broad s, 1H)

LC-MS-DAD-ELSD: Tr (min)=2.23; MH⁽⁺⁾=339⁽⁺⁾; purity: 95%

### 9.1.4: N-[(1S,2R)-1-Benzyl-2-hydroxy-3-{[3-(trifluoromethyl)benzyl]amino}-propyl]-1-oxo-2-(1-propylbutyl)-1,2,3,4-tetrahydropyrrolo[1,2-a]pyrazine-6-carboxamide

0.566 cm³ of N,N-diisopropylethylamine are poured into a suspension of 230 mg of 1-oxo-2-(1-propyl butyl)-1,2,3,4-tetrahydropyrrolo[1,2-a]pyrazine-6-carboxylic acid, 340 mg of (2R,3S)-3-amino-4-phenyl-1-{[3-(trifluoromethyl)benzyl]amino}butan-2-ol hydrochloride, 17 mg of hydroxybenzotriazole and 198 mg of 1-(3-dimethylaminopropyl)-3-ethylcarbodiimide hydrochloride in 23 cm³ of dichloromethane. The solution is kept stirring for 24 h at 20° C. 15 cm³ of water are added to the reaction medium. The aqueous phase is extracted with 15 cm³ of dichloromethane. The organic phases are combined, washed with 10 cm³ of a saturated aqueous solution of sodium chloride, dried over sodium sulfate and concentrated using a rotary evaporator under reduced pressure (5 kPa). The 840 mg of product obtained are purified by flash chromatography over silica (column: 35 g; particle size: 20-40 μm, spherical; eluant: 100% dichloromethane to 95% dichloromethane/5% methanol gradient). After concentrating the fractions under reduced pressure, 237 mg of N-[(1S,2R)-1-benzyl-2-hydroxy-3-{[3-(trifluoromethyl)benzyl]amino}propyl]-1-oxo-2-(1-propyl butyl)-1,2,3,4-tetrahydropyrrolo[1,2-a]pyrazine-6-carboxamide are obtained in the form of a foam.

LC-MS-DAD-ELSD: 599⁽⁺⁾=(M+H)⁽⁺⁾

NMR: 0.84 (t, J=7.3 Hz, 3H) 0.85 (t, J=7.3 Hz, 3H) 1.10-1.28 (m, 4H) 1.36 (m, 2H) 1.46 (m, 2H) 2.55 (partially masked m, 1H) 2.65 (m, 1H) 2.74 (dd, J=13.8, 10.7 Hz, 1H) 3.05 (dd, J=13.7, 3.8 Hz, 1H) 3.37 (t, J=5.9 Hz, 2H) 3.62 (broad m, 1H) 3.80 (s, 2H) 4.06 (m, 1H) 4.21-4.39 (m, 2H) 4.54 (m, 1H) 4.97 (broad d, J=5.0 Hz, 1H) 6.58 (d, J=4.0 Hz, 1H) 6.72 (d, J=4.0 Hz, 1H) 7.12 (m, 1H) 7.21 (m, 4H) 7.50 (t, J=7.5 Hz, 1H) 7.55 (d, J=7.5 Hz, 1H) 7.62 (d, J=7.5 Hz, 1H) 7.68 (s, 1H) 8.10 (d, J=9.0 Hz, 1H).

### 9.2: Salt

### N-[(1S,2R)-1-Benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide hydrochloride (1:1)

At a temperature close to 20° C., 230 mg of N-[(1S,2R)-1-benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide are dissolved in 3 cm³ of ethyl ether. 0.7 cm³ of a 4M hydrochloric acid solution in dioxane is added while stirring under argon, at a temperature of 5° C. The reaction mixture precipitates. The stirring is continued for 10 min, then is stopped in order to remove the supernatant. 5 cm³ of ethyl ether are again added. This operation is carried out three times. The last suspension is then concentrated under reduced pressure (5 kPa). 218 mg of N-[(1S,2R)-1-benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide hydrochloride (1:1) are obtained in the form of a beige solid.

MP: 168° C.

NMR: 0.84 (t, J=7.5 Hz, 3H); 0.86 (t, J=7.5 Hz, 3H); from 1.10 to 1.58 (m, 8H); 2.80 (dd, J=11.0 and 14.0 Hz, 1H); 2.86 (m, 1H); 3.09 (m, 1H); 3.15 (dd, J=3.5 and 14.0 Hz, 1H); 3.40 (partially masked m, 2H); 3.88 (m, 1H); 4.00 (m, 1H); from 4.22 to 4.42 (m, 4H); 4.55 (m, 1H); 5.90 (broad unresolved m, 1H); 6.62 (d, J=4.0 Hz, 1H); 6.82 (d, J=4.0 Hz, 1H); 7.13 (m, 1H); 7.23 (m, 4H); 7.65 (t, J=7.5 Hz, 1H); 7.76 (d, J=7.5 Hz, 1H); 7.86 (d, J=7.5 Hz, 1H); 7.97 (s, 1H); 8.22 (d, J=9.0 Hz, 1H); 8.99 (broad unresolved m, 1H); 9.38 (broad unresolved m, 1H).

LC-MS-DAD-ELSD: 597⁽⁻⁾=(M−H)⁽⁻⁾; 599⁽⁺⁾=(M+H)⁽⁺⁾

### EXAMPLE 10

### 10.1: Base

### N-[(1S,2R)-1-Benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide

### 10.1.1: Ethyl 1-{2-[(1-ethylpropyl)amino]ethyl}-1H-pyrrole-2-carboxylate

3.15 g of ethyl 1-(2-bromoethyl)-1H-pyrrole-2-carboxylate, 6 cm³ of 3-pentylamine, 212 mg of potassium iodide and 63 cm³ of acetonitrile are stirred at a temperature close to 20° C. under an inert atmosphere for 72 h. The mixture is then heated for 7 h at 70° C., then it is stirred for 48 h at a temperature close to 20° C. The reaction mixture is concentrated using a rotary evaporator under reduced pressure (5 kPa). 80 cm³ of dichloromethane and 50 cm³ of water are then added to the concentration residue. The phases are stirred for 10 min, then are separated. The aqueous phase is extracted with two lots of 50 cm³ of dichloromethane. The organic phases are combined, washed with 40 cm³ of a saturated aqueous solution of sodium chloride, dried over magnesium sulfate and concentrated using a rotary evaporator under reduced pressure (5 kPa). The 2.85 g of product obtained are purified by flash chromatography over silica (column: 200 g; particle size: 15-40 μm; eluant: 50% cyclohexane/50% ethyl acetate). After concentrating the fractions under reduced pressure, 1.66 g of ethyl 1-{2-[(1-ethylpropyl)amino]ethyl}-1H-pyrrole-2-carboxylate are obtained.

NMR: 0.76 (t, J=7.5 Hz, 6H); 1.26 (t, J=7.5 Hz, 3H); 1.27 (partially masked m, 4H); 1.64 (m, 1H); 2.25 (m, 1H); 2.77 (t, J=6.5 Hz, 2H); 4.19 (q, J=7.5 Hz, 2H); 4.30 (t, J=6.5 Hz, 2H); 6.08 (dd, J=2.5 and 4.0 Hz, 1H); 6.83 (dd, J=2.0 and 4.0 Hz, 1H); 7.13 (t, J=2.5 Hz, 1H).

MS-EI: M⁽⁺⁾.=252⁽⁺⁾.; IC: (M+H)⁽⁺⁾=253⁽⁺⁾

### 10.1.2: 2-(1-Ethylpropyl)-3,4-dihydropyrrolo[1,2-a]pyrazin-1(2H)-one

At a temperature close to 20° C., 1.6 g of ethyl 1-{2-[(1-ethylpropyl)amino]-ethyl}-1H-pyrrole-2-carboxylate is dissolved, under an inert atmosphere, in 60 cm³ of toluene. 6.35 cm³ of a 2M toluene solution of trimethylaluminum are poured over the reaction mixture over 5 min. The reaction mixture is heated, with stirring, for 18 h at 100° C. then is cooled to 20° C. It is then poured over an ice/water/ethyl acetate mixture. The aqueous phase is extracted with three lots of 40 cm³ of ethyl acetate. The organic phases are combined, then washed with 40 cm³ of a saturated aqueous solution of sodium chloride, dried over magnesium sulfate and concentrated using a rotary evaporator under reduced pressure (5 kPa). The crude product obtained (1.55 g) is purified by flash chromatography over silica (column: 70 g; particle size: 15-40 μm; eluant: 50% cyclohexane/50% ethyl acetate). After concentrating the fractions under reduced pressure, 0.43 g of 2-(1-ethylpropyl)-3,4-dihydropyrrolo[1,2-a]pyrazin-1(2H)-one is obtained.

MS-EI: 206⁽⁺⁾=M⁽⁺⁾

NMR: 0.94 (t, J=7.5 Hz, 6H) 1.39-1.56 (m, 4H) 3.43 (m, 2H) 4.12 (m, 2H) 4.36 (m, 1H) 6.13 (dd, J=3.8, 2.5 Hz, 1H) 6.60 (dd, J=3.8, 1.5 Hz, 1H) 6.95 (dd, J=2.5, 1.5 Hz, 1H)

### 10.1.3: 6-Bromo-2-(1-ethylpropyl)-3,4-dihydropyrrolo[1,2-a]pyrazin-1(2H)-one

At a temperature close to 20° C., 0.42 g of 2-(1-ethylpropyl)-3,4-dihydropyrrolo[1,2-a]pyrazin-1(2H)-one is dissolved in 42 cm³ of carbon tetrachloride. 363 mg of N-bromosuccinimide are added to the solution. The stirring is continued for 18 h at ambient temperature. The reaction mixture is concentrated using a rotary evaporator under reduced pressure (5 kPa). The solid residue obtained is purified by flash chromatography over silica (column: 90 g; particle size: 15-40 μm; eluant: 90% dichloromethane/10% ethyl acetate). After concentrating the fractions under reduced pressure, 0.5 g of 6-bromo-2-(1-ethylpropyl)-3,4-dihydropyrrolo[1,2-a]pyrazin-1(2H)-one is obtained.

MS-EI: 284⁽⁺⁾⁷⁹Br=M⁽⁺⁾

NMR: 0.79 (t, J=7.3 Hz, 6H) 1.38-1.57 (m, 4H) 3.49 (m, 2H) 4.04 (m, 2H) 4.33 (m, 1H) 6.31 (d, J=3.9 Hz, 1H) 6.69 (d, J=3.9 Hz, 1H)

### 10.1.4: 2-(1-Ethyl propyl)-1-oxo-1,2,3,4-tetrahydropyrrolo[1,2-a]pyrazine-6-carboxylic acid

Introduced successively, at a temperature close to 20° C., into a three-necked flask which is stirred and purged with carbon monoxide are 490 mg of 6-bromo-2-(1-ethylpropyl)-3,4-dihydropyrrolo[1,2-a]pyrazin-1(2H)-one, 15 cm³ of dimethylformamide, 0.75 cm³ of water, 0.641 g of potassium acetate, 285 mg of potassium iodide, 39 mg of palladium acetate and 91 mg of triphenylphosphine. The reaction mixture is subjected to a carbon monoxide bubbling, then is heated at 100° C. for 6 h 30 min. The reaction mixture is cooled to 20° C. and stirred for 20 h. The dimethylformamide is evaporated using a rotary evaporator under reduced pressure (5 kPa). The oily residue obtained is taken up in 80 g of ice and 80 cm³ of ethyl acetate. The pH is alkalinized with 15 cm³ of 1M sodium hydroxide. After decantation, the aqueous phase is washed with 40 cm³ of ethyl acetate. It is then acidified, with stirring, with a solution of 5M hydrochloric acid (pH=1) then extracted with three lots of 40 cm³ of ethyl acetate. The organic phases are combined, then washed with 30 cm³ of a saturated aqueous solution of sodium chloride, dried over magnesium sulfate and concentrated using a rotary evaporator under reduced pressure (5 kPa). 0.31 g of 2-(1-ethylpropyl)-1-oxo-1,2,3,4-tetrahydropyrrolo[1,2-a]pyrazine-6-carboxylic acid is obtained.

MS-EI: 250⁽⁺⁾=M⁽⁺⁾

NMR: 0.80 (t, J=7.4 Hz, 6H) 1.35-1.60 (m, 4H) 3.49 (m, 2H) 4.35 (m, 1H) 4.51 (m, 2H) 6.67 (d, J=4.1 Hz, 1H) 6.82 (d, J=4.1 Hz, 1H) 12.55 (broad unresolved m, 1H)

### 10.1.5: N-[(1S,2R)-1-Benzyl-2-hydroxy-3-{[3-(trifluoromethyl)benzyl]amino}-propyl]-2-(1-ethylpropyl)-1-oxo-1,2,3,4-tetrahydropyrrolo[1,2-a]pyrazine-6-carboxamide

Poured into a suspension of 150 mg of 2-(1-ethylpropyl)-1-oxo-1,2,3,4-tetrahydropyrrolo[1,2-a]pyrazine-6-carboxylic acid, 247 mg of (2R,3S)-3-amino-4-phenyl-1-{[3-(trifluoromethyl)benzyl]amino}butan-2-ol hydrochloride, 12.2 mg of hydroxybenzotriazole and 144 mg of 1-(3-dimethylaminopropyl)-3-ethylcarbodiimide in 15 cm³ of dichloromethane is 0.410 cm³ of N,N-diisopropylethylamine at a temperature close to 20° C. The solution is kept stirring for 72 h. 30 cm³ of dichloromethane and 15 cm³ of water are added to the reaction medium. The aqueous phase is extracted with 15 cm³ of dichloromethane. The organic phases are combined, washed with 10 cm³ of a saturated aqueous solution of sodium chloride, dried over sodium sulfate and concentrated using a rotary evaporator under reduced pressure (5 kPa). The 520 mg of product obtained are purified by flash chromatography over silica (column: 70 g; particle size: 20-40 μm, spherical; eluant: 95% dichloromethane/5% methanol then 90% dichloromethane/10% methanol). After concentrating the fractions under reduced pressure, 163 mg of N-[(1S,2R)-1-benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide are obtained.

LC-MS-DAD-ELSD: 571⁽⁺⁾=(M+H)⁽⁺⁾

NMR: 0.77 (broad t, J=7.3 Hz, 6H) 1.37-1.56 (m, 4H) 2.52-2.80 (m, 3H) 3.05 (dd, J=13.9, 3.6 Hz, 1H) 3.37 (t, J=6.0 Hz, 2H) 3.62 (broad m, 1H) 3.80 (s, 2H) 4.07 (m, 1H) 4.22-4.44 (m, 3H) 4.96 (broad d, J=4.8 Hz, 1H) 6.58 (d, J=4.1 Hz, 1H) 6.72 (dd, J=4.1 Hz, 1H) 7.12 (m, 1H) 7.21 (m, 4H) 7.45-7.58 (m, 2H) 7.62 (broad d, J=7.6 Hz, 1H) 7.69 (broad s, 1H) 8.09 (d, J=8.7 Hz, 1H)

### 10.2: Salt

### N-[(1S,2R)-1-Benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide hydro-chloride (1:1).

At a temperature close to 20° C., 155 mg of N-[(1S,2R)-1-benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide are dissolved in 1.5 cm³ of ethyl ether. 0.5 cm³ of a 4M hydrochloric acid solution in dioxane is added, while stirring under argon, at a temperature of 5° C. The reaction mixture precipitates. 5 cm³ of ethyl ether are added. The stirring is continued for 10 min, then is stopped in order to remove the supernatant. 2 cm³ of ethyl ether are again added, then removed. This operation is carried out three times. The last suspension is then concentrated under reduced pressure (5 kPa). 162 mg of N-[(1S,2R)-1-benzyl-2-hydroxy-3-{[3-(trifluoromethyl)-benzyl]amino}propyl]-2-(1-ethylpropyl)-1-oxo-1,2,3,4-tetrahydropyrrolo[1,2-a]pyrazine-6-carboxamide hydrochloride (1:1) are obtained in the form of a white solid.

MP: 178° C.

NMR: 0.77 (t, J=7.5 Hz, 3H) 0.78 (t, J=7.5 Hz, 3H) 1.47 (m, 4H); 2.79 (dd, J=11.0 and 14.0 Hz, 1H) 2.86 (m, 1H); 3.10 (m, 1H); 3.15 (dd, J=3.0 and 14.0 Hz, 1H); 3.41 (partially masked m, 2H); 3.88 (m, 1H); 4.01 (m, 1H); from 4.22 to 4.44 (m, 5H); 5.90 (very broad unresolved m, 1H); 6.63 (d, J=4.0 Hz, 1H); 6.82 (d, J=4.0 Hz, 1H); 7.14 (m, 1H); 7.23 (m, 4H); 7.65 (t, J=7.5 Hz, 1H); 7.76 (d, J=7.5 Hz, 1H); 7.85 (d, J=7.5 Hz, 1H); 7.97 (s, 1H); 8.21 (d, J=9.0 Hz, 1H); 8.97 (broad unresolved m, 1H); 9.33 (broad unresolved m, 1H).

LC-MS-DAD-ELSD: 569⁽⁺⁾=(M+H)⁽⁻⁾; 615″=(M+formic acid-H)⁽⁻⁾; 571⁽⁺⁾=(M+H)⁽⁺⁾

### EXAMPLE 11

### 11.1: Base N-[(1S,2R)-1-Benzyl-2-hydroxy-3-{[3-(trifluoromethyl)benzyl]-amino}propyl]-1-oxo-2-(1-propylbutyl)-2,3,4,5-tetrahydro-1H-pyrrolo[1,2-a]-[1,4]diazepine-7-carboxamide

### 11.1.1: Ethyl 1-(3-bromopropyl)-1H-pyrrole-2-carboxylate

5.15 g of ethyl pyrrole-2-carboxylate, 18 cm³ of 1,3-dibromopropane, 22 cm³ of concentrated sodium hydroxide and 11.93 g of tetrabutylammonium bromide are stirred under an inert atmosphere for 48 h at a temperature close to 20° C. 80 cm³ of dichloromethane and 80 cm³ of water are added to the reaction medium. The aqueous phase is extracted with three lots of 40 cm³ of dichloromethane. The organic phases are combined, then washed with 50 cm³ of a saturated aqueous solution of sodium chloride, dried over magnesium sulfate and concentrated using a rotary evaporator under reduced pressure (5 kPa). The 50 g of oil obtained are purified by flash chromatography over silica (column: 400 g; particle size: 15-40 μm; eluant: 100% dichloromethane). After concentrating the fractions under reduced pressure, 9.8 g of ethyl 1-(3-bromopropyl)-1H-pyrrole-2-carboxylate are obtained.

MS: 259⁽⁺⁾⁷⁹Br=M⁽⁺⁾

1H NMR (400 MHz, DMSO-d₆) d ppm 1.27 (t, J=7.1 Hz, 3H) 2.22 (m, 2H) 3.40 (t, J=6.7 Hz, 2H) 4.21 (q, J=7.1 Hz, 2H) 4.38 (t, J=6.7 Hz, 2H) 6.12 (dd, J=3.9, 2.5 Hz, 1H) 6.87 (dd, J=3.9, 2.0 Hz, 1H) 7.14 (dd, J=2.5, 2.0 Hz, 1H)

### 11.1.2: Ethyl 1-{3-[(1-propylbutyl)amino]propyl}-1H-pyrrole-2-carboxylate

7 g of ethyl 1-(3-bromopropyl)-1H-pyrrole-2-carboxylate, 9.3 g of 4-aminoheptane, 4.46 g of potassium iodide and 140 cm³ of acetonitrile are stirred under an inert atmosphere for 20 h at 70° C., then cooled to a temperature close to 20° C. The reaction mixture is concentrated using a rotary evaporator under reduced pressure (5 kPa). 100 cm³ of dichloromethane and 100 cm³ of water are then added to the concentration residue. The aqueous phase is extracted with three lots of 50 cm³ of dichloromethane. The organic phases are combined, washed with 40 cm³ of a saturated aqueous solution of sodium chloride, dried over magnesium sulfate and concentrated using a rotary evaporator under reduced pressure (5 kPa). The product obtained is purified by flash chromatography over silica (column: 400 g; particle size: 15-40 μm; eluant: 75% heptane/25% ethyl acetate to 100% ethyl acetate gradient). After concentrating the fractions under reduced pressure, 5.87 g of ethyl 1-{3-[(1-propylbutyl)amino]propyl}-1H-pyrrole-2-carboxylate are obtained.

MS-EI: 294⁽⁺⁾=M⁽⁺⁾; 251⁽⁺⁾=M⁽⁺⁾-C₃H₇

1H NMR (300 MHz, DMSO-d₆) d ppm 0.85 (t, J=6.9 Hz, 6H) 1.13-1.35 (m, 11H) 1.76 (m, 2H) 2.28-2.44 (m, 3H) 4.19 (q, J=7.1 Hz, 2H) 4.32 (t, J=6.9 Hz, 2H) 6.08 (dd, J=2.5, 1.8 Hz, 1H) 6.83 (dd, J=3.9, 1.8 Hz, 1H) 7.10 (dd, J=2.5, 1.9 Hz, 1H)

### 11.1.3: 2-(1-Propylbutyl)-2,3,4,5-tetrahydro-1H-pyrrolo[1,2-a][1,4]diazepin-1-one

At a temperature close to 20° C., 5.85 g of ethyl 1-{3-[(1-propylbutyl)amino]-propyl}-1H-pyrrole-2-carboxylate are dissolved under an inert atmosphere in 150 cm³ of toluene. 29.8 cm³ of a 2M toluene solution of trimethylaluminum are poured over the reaction mixture over 5 min. The reaction mixture is heated, with stirring, for 20 h at 100° C., then is cooled to a temperature close to 20° C. It is then poured over 200 g of ice and 100 cm³ of ethyl acetate. The suspension obtained is filtered over a Celite 545 pellet. The aqueous phase is extracted with three lots of 40 cm³ of ethyl acetate. The organic phases are combined, then washed with 40 cm³ of water, 40 cm³ of a saturated aqueous solution of sodium chloride, dried over magnesium sulfate and concentrated using a rotary evaporator under reduced pressure (5 kPa). The 4.71 g of crude product obtained are purified by flash chromatography over silica (column: 200 g; particle size: 15-40 μm; eluant: 100% dichloromethane to 80% dichloromethane/20% ethyl acetate gradient). After concentrating the fractions under reduced pressure, 2.97 g of 2-(1-propylbutyl)-2,3,4,5-tetrahydropyrrolo[1,2-a][1,4]-diazepin-1-one are obtained.

MS-EI: 248⁽⁺⁾=M⁽⁺⁾; 249⁽⁺⁾=(M+H)⁽⁺⁾

1H NMR (300 MHz, DMSO-d₆) d ppm 0.88 (t, J=7.3 Hz, 6H) 1.13-1.34 (m, 4H) 1.35-1.58 (m, 4H) 1.98 (m, 2H) 3.13 (t, J=6.7 Hz, 2H) 4.11 (t, J=6.7 Hz, 2H) 4.46 (m, 1H) 6.02 (m, 1H) 6.48 (m, 1H) 6.88 (broad s, 1H)

### 11.1.4: 7-Bromo-2-(1-propylbutyl)-2,3,4,5-tetrahydro-1H-pyrrolo[1,2-a][1,4]diazepin-1-one

At a temperature close to 20° C., 1.5 g of 2-(1-propylbutyl)-2,3,4,5-tetrahydro-1H-pyrrolo[1,2-a][1,4]diazepin-1-one are dissolved in 120 cm³ of carbon tetrachloride. 1.075 g of N-bromosuccinimide is added to the solution. The stirring is continued for 3 h. The reaction mixture is concentrated using a rotary evaporator under reduced pressure (5 kPa). The solid residue obtained is purified by flash chromatography over silica (column: 200 g; particle size: 15-40 μm; eluant: 90% dichloromethane/10% ethyl acetate). After concentrating the fractions under reduced pressure, 1.92 g of 7-bromo-2-(1-propylbutyl)-2,3,4,5-tetrahydro-1H-pyrrolo[1,2-a][1,4]diazepin-1-one are obtained.

MS-EI: 326⁽⁺⁾⁷⁹Br=M⁽⁺⁾

1H NMR (300 MHz, DMSO-d₆) d ppm 0.88 (t, J=7.2 Hz, 6H) 1.12-1.33 (m, 4H) 1.35-1.56 (m, 4H) 1.97 (m, 2H) 3.12 (t, J=6.7 Hz, 2H) 4.11 (t, J=6.7 Hz, 2H) 4.45 (m, 1H) 6.23 (d, J=3.9 Hz, 1H) 6.56 (d, J=3.9 Hz, 1H)

### 11.1.5: 1-Oxo-2-(1-propylbutyl)-2,3,4,5-tetrahydro-1H-pyrrolo[1,2-a][1,4]-diazepine-7-carboxylic acid

Introduced successively, at a temperature close to 20° C., into a three-necked flask which is stirred and purged with carbon monoxide are 1 g of 7-bromo-2-(1-propylbutyl)-2,3,4,5-tetrahydro-1H-pyrrolo[1,2-a][1,4]-diazepin-1-one, 25 cm³ of dimethylformamide, 2.5 cm³ of water, 1.14 g of potassium acetate, 102 mg of potassium iodide, 275 mg of palladium acetate and 640 mg of triphenylphosphine. The reaction mixture is subjected to a carbon monoxide bubbling, then is heated at 100° C. for 7 h. It is cooled to 20° C. in order to be filtered over a Celite 545 pellet. The filtrate is evaporated using a rotary evaporator under reduced pressure (5 kPa). The oily residue obtained is taken up in 40 g of ice and 40 cm³ of ethyl acetate. The pH is brought to 10 with 5M sodium hydroxide. After decantation, the aqueous phase is washed with three lots of 40 cm³ of ethyl acetate then filtered over a Celite 545 pellet. The filtrate is acidified, with stirring, with a 5M hydrochloric acid solution (pH=1) then extracted with three lots of 40 cm³ of ethyl acetate. The organic phases are combined, then washed with 30 cm³ of a saturated aqueous solution of sodium chloride, dried over magnesium sulfate and concentrated using a rotary evaporator under reduced pressure (5 kPa). The product obtained (1 g) is purified by flash chromatography over silica (column: 90 g; particle size: 15-40 μm; eluant: 95% dichloromethane/5% methanol). After concentrating the fractions under reduced pressure, 0.74 g of 1-oxo-2-(1-propylbutyl)-2,3,4,5-tetrahydro-1H-pyrrolo[1,2-a][1,4]diazepine-7-carboxylic acid is obtained.

MS-EI: 292⁽⁺⁾=M⁽⁺⁾

1H NMR (400 MHz, DMSO-d₆) d ppm 0.88 (t, J=7.2 Hz, 6H) 1.25 (m, 4H) 1.46 (m, 4H) 1.99 (m, 2H) 3.11 (t, J=6.7 Hz, 2H) 4.51 (m, 1H) 4.62 (t, J=6.7 Hz, 2H) 6.47 (d, J=4.1 Hz, 1H) 6.79 (d, J=4.1 Hz, 1H) 12.59 (broad unresolved m, 1H)

### 1.1.6: N-[(1S,2R)-1-Benzyl-2-hydroxy-3-{[3-(trifluoromethyl)benzyl]amino}-propyl]-1-oxo-2-(1-propylbutyl)-2,3,4,5-tetrahydro-1H-pyrrolo[1,2-a][1,4]-diazepine-7-carboxamide

Poured into a suspension of 200 mg of 1-oxo-2-(1-propylbutyl)-2,3,4,5-tetrahydro-1H-pyrrolo[1,2-a][1,4]diazepine-7-carboxylic acid, 281 mg of (2R,3S)-3-amino-4-phenyl-1-{[3-(trifluoromethyl)benzyl]amino}butan-2-ol hydrochloride (1:1), 14 mg of hydroxybenzotriazole and 164 mg of 1-(3-dimethylaminopropyl)-3-ethylcarbodiimide hydrochloride in 25 cm³ of dichloromethane is 0.468 cm³ of N,N-diisopropylethylamine at a temperature close to 20° C. The solution obtained is kept stirring for 3 h 30 min at 20° C. under an inert atmosphere. 25 cm³ of dichloromethane and 15 cm³ of water are added to the reaction medium. The organic phase is washed with 20 cm³ of a saturated aqueous solution of sodium chloride, dried over sodium sulfate and concentrated using a rotary evaporator under reduced pressure (5 kPa). The product obtained is purified by flash chromatography over silica (column: 15 g; particle size: 20-40 μm, spherical; eluant: 95% dichloromethane/5% methanol to 90% dichloromethane/10% methanol gradient). After concentrating the fractions under reduced pressure, 206 mg of N-[(1S,2R)-1-benzyl-2-hydroxy-3-{[3-(trifluoromethyl)benzyl]amino}propyl]-1-oxo-2-(1-propylbutyl)-2,3,4,5-tetrahydro-1H-pyrrolo[1,2-a][1,4]diazepine-7-carboxamide are obtained.

LC-MS-DAD-ELSD: 611⁽⁻⁾=(M−H)⁽⁻⁾; 657⁽⁻⁾=(M+formic acid-H)⁽⁻⁾; 613⁽⁺⁾=(M+H)⁽⁺⁾

1H NMR: (400 MHz, DMSO-d₆) d ppm 0.86 (t, J=7.3 Hz, 3H) 0.88 (t, J=7.3 Hz, 3H) 1.15-1.30 (m, 4H) 1.34-1.51 (m, 4H) 1.86 (m, 2H) 2.46-2.77 (partially masked m, 3H) 2.93-3.14 (m, 3H) 3.61 (m, 1H) 3.73-3.86 (m, 2H) 4.03 (m, 1H) 4.22 (m, 1H) 4.35 (m, 1H) 4.47 (m, 1H) 4.98 (d, J=5.9 Hz, 1H) 6.39 (d, J=3.9 Hz, 1H) 6.54 (d, J=3.9 Hz, 1H) 7.12 (m, 1H) 7.21 (m, 4H) 7.47-7.58 (m, 2H) 7.62 (broad d, J=7.5 Hz, 1H) 7.69 (broad s, 1H) 8.08 (d, J=9.0 Hz, 1H)

### 11.2: Salt

### N-[(1S,2R)-1-Benzyl-2-hydroxy-3-{[3-(trifluoromethyl)benzyl]amino}propyl]-1-oxo-2-(1-propylbutyl)-2,3,4,5-tetrahydro-1H-pyrrolo[1,2-a][1,4]diazepine-7-carboxamide hydrochloride (1:1)

At a temperature close to 20° C., 200 mg of N-[(1S,2R)-1-benzyl-2-hydroxy-3-{[3-(trifluoromethyl)benzyl]amino}propyl]-1-oxo-2-(1-propyl butyl)-2,3,4,5-tetrahydro-1H-pyrrolo[1,2-a][1,4]diazepine-7-carboxamide are dissolved in 2.5 cm³ of ethyl ether. 0.6 cm³ of a 4M hydrochloric acid solution in dioxane are added, while stirring under argon, at a temperature of 5° C. The reaction mixture partially precipitates. The suspension is then concentrated under reduced pressure (5 kPa). 3 cm³ of ethyl ether are added. The suspension is stirred for 15 min, then the stirring is stopped and the supernatant is removed. This operation is carried out two times. The last suspension is then concentrated under reduced pressure (5 kPa). 183 mg of N-[(1S,2R)-1-benzyl-2-hydroxy-3-{[3-(trifluoromethyl)benzyl]amino}-propyl]-1-oxo-2-(1-propyl butyl)-2,3,4,5-tetrahydro-1H-pyrrolo[1,2-a][1,4]-diazepine-7-carboxamide hydrochloride (1:1) are obtained in the form of a white solid.

1H NMR (400 MHz, DMSO-d₆) d ppm 0.86 (t, J=7.5 Hz, 3H) 0.88 (t, J=7.5 Hz, 3H); from 1.15 to 1.30 (m, 4H); 1.44 (m, 4H); 1.88 (m, 2H); 2.77 (dd, J=11.0 and 14.0 Hz, 1H); 2.87 (m, 1H); from 2.96 to 3.17 (m, 4H); 3.87 (m, 1H); 3.97 (m, 1H); 4.17 (m, 1H); 4.31 (m, 2H); from 4.41 to 4.53 (m, 2H); 5.92 (broad unresolved m, 1H); 6.44 (d, J=4.0 Hz, 1H); 6.62 (d, J=4.0 Hz, 1H); 7.14 (m, 1H); 7.24 (m, 4H); 7.65 (t, J=7.5 Hz, 1H); 7.76 (d, J=7.5 Hz, 1H); 7.86 (d, J=7.5 Hz, 1H); 7.96 (s, 1H); 8.18 (d, J=9.0 Hz, 1H); 8.97 (broad unresolved m, 1H); 9.32 (broad unresolved m, 1H)

LC-MS-DAD-ELSD: 611⁽⁻⁾=(M−H)⁽⁻⁾; 657⁽⁻⁾=(M+formic acid-H)⁽⁻⁾; 613⁽⁺⁾=(M+H)⁽⁺⁾

Table 1 below illustrates the chemical structures and the physical properties of some examples of compounds according to the invention. In this table:
MP (° C.) represents the melting point of the compound in degrees Celsius;
in the “salt” column, “−” represents a compound in the free base form, whereas “HCl” represents a compound in the hydrochloride form, the ratio between parentheses is the (acid:base) ratio;
R3 represents a trifluoromethyl group;
Me and Et represent, respectively, methyl and ethyl groups; and
“nd”: not determined.

The compounds described in this table were prepared according to the methods described previously.

TABLE 1

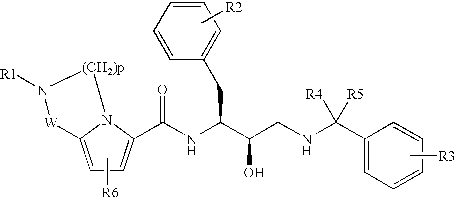

| Compound | R1 | W | p | R2 | R4, R5 | R6 | MP (° C.) | Salt |
|---|---|---|---|---|---|---|---|---|
| 1 | BOC— | —CH₂— | 2 | H— | —CH₂CH₂— | 8-COOMe | 136 | — |
| 2 | BOC— | —CH₂— | 2 | H— | —CH₂CH₂— | 8-COOH | 134.5 | — |
| 3 | H— | —CH₂— | 2 | H— | —CH₂CH₂— | 8-COOH | 196 | HCl (2:1) |
| 4 | BOC— | —CH₂— | 2 | H— | —CH₂CH₂— | 8-CON(Et)₂ | nd^{(a)} | — |
| 5 | H— | —CH₂— | 2 | H— | —CH₂CH₂— | 8-CON(Et)₂ | 143 | HCl (2:1) |
| 6 | MeSO₂— | —CH₂— | 2 | H— | —CH₂CH₂— | 8-CON(Et)₂ | nd^{(b)} | — |
| 7 | (n-C₃H₇)₂CH— | C═O | 2 | 3,5-diF | —CH₂CH₂— | H— | 183 | HCl (1:1) |
| 8 | (n-C₃H₇)₂CH— | C═O | 2 | H— | —CH₂CH₂— | H— | 176 | HCl (1:1) |
| 9 | (n-C₃H₇)₂CH— | C═O | 2 | H— | H—, H— | H— | 168 | HCl (1:1) |
| 10 | Et₂CH— | C═O | 2 | H— | H—, H— | H— | 178 | HCl (1:1) |
| 11 | Et₂CH | C═O | 3 | H— | H—, H— | H— | nd^{(a)} | HCl (1:1) |

^{(a)}characterized by a ¹H NMR spectrum and by liquid chromatography coupled to a mass spectrometer
^{(b)}characterized by a ¹H NMR spectrum

The compounds according to the invention have been the subject of pharmacological tests enabling their inhibitory effect with respect to β-secretase activity to be determined.

The tests consisted in measuring the in vitro inhibition of the β-secretase activity by the compounds of the invention.

The β-secretase activity measured corresponds to that of a purified recombinant form of human BACE1 aspartyl protease (the latter comprising a hexahistidine tag at the C-terminal position) produced by expression in Drosophila cells. The purified enzyme is conditioned in TRIS buffer (18 mM) at pH 7.5 containing NaCl (0.45M), MnCl₂ (0.9 mM), CaCl₂ (0.9 mM), α-D-methylmannoside and 10% glycerol, and stored at −80° C. until use.

The BACE1 activity is measured from the cleavage of a fluorogenic peptide substrate, known as FS1, described originally by Ermolieff et Coll. (2000, Biochemistry, 39, 12450-12456), and based on the principle of fluorescence resonance energy transfer (FRET); the cleavage of the FS1 peptide is measured from the increase of the fluorescent signal emitted by the EDANS (or 5-[(2-aminoethyl)amino]-naphthalene-2-sulfonic acid) group.

The test is carried out in a 96-well microplate in order to determine the inhibition of the enzyme activity by the products of the invention. The substrate FS1 is solubilized to a concentration of 1 mM in 100% dimethylsulfoxide (DMSO) and stored at −20° C. until use. The dilutions of the products to be tested are prepared in DMSO starting from a 10 mM stock solution. The products of the invention, at final concentrations of 0.003 to 10 μM are incubated at 37° C. with the substrate FS1 (final concentration of 5 μM) and the purified enzyme (final concentration of 10 nM) in the sodium acetate buffer (0.1 M), pH 4.5 containing 0.02% of CHAPS detergent and 200 nM NaCl for 45 minutes. The final percentage of DMSO does not exceed 7%. When the incubation is finished, the fluorescence is measured in a spectrofluorimeter, at the excitation wavelength of 355 nm and emission wavelength of 509 nm. For each product concentration tested, the fluorescent signal is compared to the maximum signal obtained when the substrate FS1 is only incubated with the enzyme.

The inhibitory activity of the products of the invention is then evaluated by the measurement of the IC₅₀ (concentration of product giving 50% inhibition of the enzyme activity) using a non-linear regression analysis (computer application software XLfit, IDBS™).

The IC₅₀ values are between 0.1 and 5 μM.

For example, compounds No. 5, 7, 9 and 11 showed an IC₅₀ of 0.31; 0.48; 0.37 and 1.40 μM respectively.

It therefore appears that the compounds according to the invention have an inhibitory activity with respect to the activity of 8-secretase.

The compounds according to the invention may therefore be used for the preparation of medicaments, in particular medicaments that inhibit the production of Aβ.

Thus, another subject of the invention is, according to another of its aspects, medicaments which comprise a compound of formula (I) or an addition salt of the latter with a pharmaceutically acceptable acid, or else a hydrate or a solvate of the compound of formula (I).

These medicaments find their use in therapeutics, especially in the treatment and prevention of diseases associated with the production of the Aβ peptide, among which mention may be made of neurodegenerative diseases such as Alzheimer's disease, Parkinson's disease, Huntington's disease, Creutzfeldt-Jakob disease, Down's syndrome, dementia with Lewy bodies, senile dementia, frontotemporal dementia, cerebral and systemic amyloidosis, mild cognitive impairments, cerebral amyloid angiopathy, primary and secondary memory disorders, amyotrophic lateral sclerosis, multiple sclerosis, peripheral neuropathies, diabetic neuropathies, migraine, mood disorders, depression, anxiety, vascular disorders such as atherosclerosis, cerebrovascular ischemia, tumors and cell proliferation disorders.

These medicaments find, in particular, their use in the treatment and the prevention of neurodegenerative diseases such as Alzheimer's disease, Parkinson's disease, Down's syndrome, dementia with Lewy bodies, senile dementia, frontotemporal dementia, cerebral and systemic amyloidosis, mild cognitive impairments, cerebral amyloid angiopathy, primary and secondary memory disorders and cerebrovascular ischemia.

According to another of its aspects, the present invention relates to pharmaceutical compositions comprising, as the active principle, a compound according to the invention.

These pharmaceutical compositions contain an effective dose of at least one compound according to the invention, or a pharmaceutically acceptable salt, a hydrate or solvate of said compound, and also at least one pharmaceutically acceptable excipient.

Said excipients are chosen, depending on the pharmaceutical form and the desired method of administration, from the customary excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or its optional salt, solvate or hydrate, may be administered in unit administration form, as a mixture with conventional pharmaceutical excipients, to animals and human beings for the prophylaxis or the treatment of the above disorders or diseases.

The suitable unit administration forms comprise oral forms, such as tablets, soft or hard gelatin capsules, powders, granules and/or oral solutions or suspensions, sublingual, buccal, intratracheal, intraocular and intranasal administration forms, forms for administration by inhalation, topical, transdermal, subcutaneous, intramuscular or intravenous administration forms, rectal administration forms and implants. For topical application it is possible to use the compounds according to the invention in creams, gels, ointments or lotions.

By way of example, a unit administration form of a compound according to the invention in tablet form may comprise the following components:

| | |
|---|---|
| Compound according to the invention | 50.0 mg |
| Mannitol | 223.75 mg |
| Croscarmellose sodium | 6.0 mg |
| Maize starch | 15.0 mg |
| Hydroxypropyl methyl cellulose | 2.25 mg |
| Magnesium stearate | 3.0 mg |

The present invention, according to another of its aspects, also relates to a method for treating the pathologies indicated above which comprises the administration, to a patient, of an effective dose of a compound according to the invention, or one of its pharmaceutically acceptable salts or hydrates or solvates.

We claim:

1. A compound of formula (I) 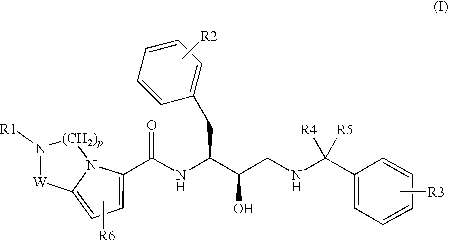
wherein:
R1 is hydrogen, (C₁-C₁₀)alkyl, (C₃-C₇)cycloalkyl, (CH₂)ₙ—(C₁-C₆)alkenyl, (CH₂)ₙ—(C₁-C₆)alkynyl, (C₁-C₆)alkyl-Z—(C₁-C₆)alkyl, COOR, S(O)ₘR, aryl or aralkyl, wherein the (C₁-C₁₀)alkyl, (C₃-C₇)cycloalkyl, (CH₂)ₙ—(C₁-C₆)alkenyl, (CH₂)ₙ—(C₁-C₆)alkynyl, (C₁-C₆)alkyl-Z—(C₁-C₆)alkyl, aryl and aralkyl are optionally substituted with one or more groups chosen from halogen, (C₁-C₆)alkyl, (C₃-C₇)cycloalkyl, halo(C₁-C₆)alkyl, (C₁-C₆)alkoxy, halo(C₁-C₆)alkoxy, NR7R8, nitro, cyano, OR, COOR, C(O)NR7R8 and S(O)ₘNR7R8;
R2 is one or more groups chosen from hydrogen, halogen, (C₁-C₆)alkyl, (C₃-C₇)cycloalkyl, (C₁-C₆)alkenyl, (C₁-C₆)alkynyl, (C₁-C₆)alkyl-Z—(C₁-C₆)alkyl, halo(C₁-C₆)alkyl, halo(C₁-C₆)alkoxy, hydroxy, (C₁-C₆)alkoxy, nitro, cyano, amino, NR7R8, COOR, C(O)NR7R8, O—C(O)(C₁-C₆)alkyl, S(O)ₘ—NR7R8, and aryl, wherein the aryl is optionally substituted with one or more groups chosen from halogen, (C₁-C₆)alkyl, (C₃-C₇)cycloalkyl, halo(C₁-C₆)alkyl, (C₁-C₆)alkoxy, halo(C₁-C₆)alkoxy, NR7R8, OR, nitro, cyano, COOR, C(O)NR7R8 and S(O)ₘNR7R8;
R3 is trifluoromethyl;
R4 and R5 are hydrogen, or
R4 and R5 taken together with the carbon atom to which they are attached for a saturated ring containing from 3 to 6 carbon atoms and optionally containing from 0 to 1 heteroatom, chosen from O, N and S;
R6 is hydrogen, halogen, (C₁-C₆)alkyl, (C₃-C₇)cycloalkyl, (C₃-C₇)cycloalkyl(C₁-C₆)alkyl, nitro, amino, NR7R8, COOR, aryl, NR7(SO₂)R8 or C(O)NR7R8;
R, R7 and R8 are, independently of one another, hydrogen, (C₁-C₆)alkyl, (C₃-C₇)cycloalkyl, (C₃-C₇)cycloalkyl(C₁-C₆)alkyl, aryl, or aryl(C₁-C₆)alkylene, or
R7 and R8 taken together with the nitrogen atom to which they are attached form a saturated, partially unsaturated or unsaturated ring containing from 5 to 7 carbon atoms and optionally containing, in addition, a heteroatom chosen from O, N and S(O)ₘ;
W is methylene or C(O);
Z is O, N or S(O)ₘ;
m is 0, 1 or 2;
n is 1, 2, 3, 4, 5 or 6; and
p is 2 or 3;
provided that the carbon bearing the benzyl group substituted by R2 is of S absolute configuration; and
the carbon bearing the hydroxyl group is of R absolute configuration;
or an addition salt with an acid thereof.

2. The compound according to claim 1, wherein W is methylene, or an addition salt with an acid thereof.

3. The compound according to claim 1, wherein W is C(O), or an addition salt with an acid thereof.

4. The compound according to claim 1, wherein p is 2, or an addition salt with an acid thereof.

5. The compound according to claim 1, wherein p is 3, or an addition salt with an acid thereof.

6. The compound according to claim 1, wherein R6 is hydrogen, COOR or C(O)NR7R8, or an addition salt with an acid thereof.

7. The compound according to claim 1, wherein R6 is hydrogen, COOH, COOMe or C(O)N(Et)₂, or an addition salt with an acid thereof.

8. The compound according to claim 1, wherein:
R1 is hydrogen, (C₁-C₁₀)alkyl, COOR or S(O)ₘR, wherein the (C₁-C₁₀)alkyl is optionally substituted with one or more (C₁-C₆)alkyl;
R2 is one or more groups chosen from hydrogen and halogen;
R4 and R5 are hydrogen, or
R4 and R5 taken together with the carbon atom to which they are attached form a cyclopropyl group;
R6 is hydrogen, COOR or C(O)NR7R8; and
R, R7 and R8 are, independently of one another, hydrogen or (C₁-C₆)alkyl;
or an addition salt with an acid thereof.

9. The compound or the addition salt with an acid according to claim 1, which is:
2-tert-butyl 8-methyl 6-{[(1S,2R)-1-benzyl-2-hydroxy-3-({1-[3-(trifluoromethyl)phenyl]cyclopropyl}amino)propyl]carbamoyl}-3,4-dihydropyrrolo[1,2-a]pyrazine-2,8(1H)-dicarboxylate;
6-{[(1S,2R)-1-benzyl-2-hydroxy-3-({1-[3-(trifluoromethyl)phenyl]cyclopropyl}amino)propyl]carbamoyl}-2-(tert-butoxycarbonyl)-1,2,3,4-tetrahydropyrrolo[1,2-a]pyrazine-8-carboxylic acid;
6-{[(1S,2R)-1-benzyl-2-hydroxy-3-({1-[3-(trifluoromethyl)phenyl]cyclopropyl}amino)propyl]carbamoyl}-1,2,3,4-tetrahydropyrrolo[1,2-a]pyrazine-8-carboxylic acid hydrochloride (2:1);
tert-butyl 6-{[(1S,2R)-1-benzyl-2-hydroxy-3-({1-[3-(trifluoromethyl)phenyl]cyclopropyl}amino)propyl]carbamoyl}-8-(diethylcarbamoyl)-3,4-dihydropyrrolo[1,2-a]pyrazine-2(1H)carboxylate;
6-{(1S,2R)-1-benzyl-2-hydroxy-3-[1-(3-trifluoromethylphenyl)cyclopropylamino]propyl}-N-8,N-8-diethyl-1,2,3,4-tetrahydropyrrolo[1,2-a]pyrazine-6,8-dicarboxamide;
methylphenyl)cyclopropylamino]propyl}-N-8,N-8-diethyl-1,2,3,4-tetrahydropyrrolo[1,2-a]pyrazine-6,8-dicarboxamide hydrochloride (2:1);
6-{(1S,2R)-1-benzyl-2-hydroxy-3-[1-(3-trifluoromethylphenyl)cyclopropylamino]propyl}-N-8,N-8-diethyl-2-(methylsulfonyl)-1,2,3,4-tetrahydropyrrolo[1,2-a]pyrazine-6,8-dicarboxamide;
N-[(1S,2R)-1-(3,5-difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide;
N-[(1S,2R)-1-(3,5-difluorobenzyl)-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide hydrochloride (1:1);
N-[(1S,2R)-1-benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide;
N-[(1S,2R)-1-benzyl-2-hydroxy-3-({1-[3-(trifluoromethyl)phenyl]cyclopropyl}amino)propyl]-1-oxo-2-(1-propylbutyl)-1,2,3,4-tetrahydropyrrolo[1,2-a]pyrazine-6-carboxamide hydrochloride (1:1);
N-[(1S,2R)-1-benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide;
N-[(1S,2R)-1-benzyl-2-hydroxy-3-{[3-(trifluoromethyl)benzyl]amino}propyl]-1-oxo-2-(1-propylbutyl)-1,2,3,4-tetrahydropyrrolo[1,2-a]pyrazine-6-carboxamide hydrochloride (1:1);
N-[(1S,2R)-1-benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide;
N-[(1S,2R)-1-benzyl-2-hydroxy-3-{[3-(trifluoromethyl)benzyl]amino}propyl]-2-(1-ethylpropyl)-1-oxo-1,2,3,4-tetrahydropyrrolo[1,2-a]pyrazine-6-carboxamide hydrochloride (1:1);
N-[(1S,2R)-1-benzyl-2-hydroxy-3-{[3-(trifluoromethyl)benzyl]amino}propyl]-1-oxo-2-(1-propylbutyl)-2,3,4,5-tetrahydro-1H-pyrrolo[1,2-a][1,4]diazepine-7-carboxamide; or
N-[(1S,2R)-1-benzyl-2-hydroxy-3-{[3-(trifluoromethyl)benzyl]amino}propyl]-1-oxo-2-(1-propylbutyl)-2,3,4,5-tetrahydro-1H-pyrrolo[1,2-a][1,4]diazepine-7-carboxamide hydrochloride (1:1).

10. A compound of formula (IIIa) 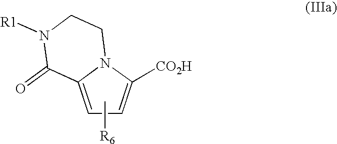
wherein R1 and R6 are as defined as claim 1.

11. A compound of formula (IIIb) 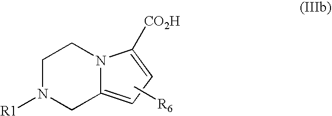
wherein R1 and R6 are as defined in claim 1.

12. The compound according to claim 11, which is 2-(tert-butoxycarbonyl)-8-(methoxycarbonyl)-1,2,3,4-tetrahydropyrrolo[1,2-a]pyrazine-6-carboxylic acid.

13. A compound of formula (IIIc) 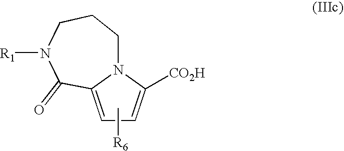
wherein R1 and R6 are as defined in claim 1.

14. A pharmaceutical composition comprising the compound according to claim 1, or an addition salt with a pharmaceutically acceptable acid thereof, in combination with at least one pharmaceutically acceptable excipient.

15. A pharmaceutical composition comprising the compound or the addition salt with an acid thereof according to claim 9, in combination with at least one pharmaceutically acceptable excipient.

16. A method for treating a disease in which beta-secretase activity is involved, in a patient in need thereof, comprising administering to the patient a pharmaceutically effective amount of the compound according to claim 1, or an addition salt with a pharmaceutically acceptable acid thereof.

17. A method for treating Alzheimer's disease, Parkinson's disease, Huntington's disease, Creutzfeldt-Jakob disease, Down's syndrome, dementia with Lewy body, senile dementia, frontotemporal dementia, cerebral or systemic amyloidosis, mild cognitive impairment, cerebral amyloid angiopathy, primary or secondary memory disorder, amyotrophic lateral sclerosis, multiple sclerosis, peripheral neuropathy, diabetic neuropathy, migraine, mood disorder, depression, anxiety, vascular disorder, atherosclerosis, cerebrovascular ischemia, tumor or cell proliferation disorder, in a patient in need thereof, comprising administering to the patient a pharmaceutically effective amount of the compound according to claim 1, or an addition salt with a pharmaceutically acceptable acid thereof.

18. A method for treating Alzheimer's disease, Parkinson's disease, Down's syndrome, dementia with Lewy body, senile dementia, frontotemporal dementia, cerebral or systemic amyloidosis, mild cognitive impairment, cerebral amyloid angiopathy, primary or secondary memory disorder, or cerebrovascular ischemia, in a patient in need thereof, comprising administering to the patient a pharmaceutically effective amount of the compound according to claim 1, or an addition salt with a pharmaceutically acceptable acid thereof.
